(19) United States
(12) Patent Application Publication
(10) Pub. No.: US 2025/0327007 A1
(43) Pub. Date: Oct. 23, 2025

(54) BIOLOGICAL CULTURE UNIT

(71) Applicant: Keratify Limited, Sidcup Kent (GB)

(72) Inventors: Zuber Ahmed, Kent (GB); Rosalind Hannen, Kent (GB)

(21) Appl. No.: 18/857,774

(22) PCT Filed: Apr. 19, 2023
(86) PCT No.: PCT/GB2023/051026

§ 371 Date: Oct. 17, 2024

(30) Foreign Application Priority Data
Apr. 19, 2022 (GB) 2205709.5

(51) Int. Cl. C12M 3/00; C12M 1/00; C12M 1/04; C12M 1/107; C12M 1/32; C12M 1/34; C12M 3/06

(52) U.S. Cl. CPC C12M 21/08; C12M 23/12; C12M 23/16; C12M 23/24; C12M 23/34; C12M 23/36; C12M 23/40; C12M 29/04; C12M 29/12; C12M 41/40

(57) ABSTRACT

Aspects and embodiments relate to a biological sample culture unit, a biological sample culture module comprising at least two biological sample culture units and culturing apparatus including a biological sample culture unit or biological sample culture module. Aspects and embodiments also provide methods to culture biological samples within a biological culture unit, biological culture module or biological culture apparatus. All aspects utilise a biological sample culture unit comprising a first chamber configured to accommodate a biological sample, culture medium and a gas reservoir; a second chamber configured to accommodate a reservoir of culture medium and a further gas reservoir, the second chamber comprising a gas port configured to couple the further gas reservoir to a gas source; an inlet conduit linking the second chamber with the first chamber configured to allow flow of fluid between the second chamber and the first chamber in dependence upon a pressure difference between the gas reservoirs in the second chamber and the first chamber. Aspects support provision of a small footprint biological sample culture environment which is highly scalable, which can sustain a culture environment with controllable conditions for an extended period and in which maintenance of, and testing a biological sample within, the culture environment can be automated, thus facilitating high throughput, minimal disruption to biological samples under study and improved reproducibility.

### FIELD OF THE INVENTION

Aspects and embodiments relate to a biological sample culture unit, a biological sample culture module comprising at least two biological sample culture units and culturing apparatus including a biological sample culture unit or biological sample culture module. Aspects and embodiments also provide methods to culture biological samples within a biological culture unit, biological culture module or biological culture apparatus. Further aspects provide a fluidic device, and methods of providing and operating such a device. Yet further aspects relate to a test plate and methods of providing and operating such a test plate.

### BACKGROUND

Cell culture devices and fluidic devices are known. Typically fluidic, including microfluidic, devices are provided for the purposes of cell and tissue culture. Fluidic culture systems can offer many benefits in relation to in vitro cell and ex vivo tissue culture. In particular, fluidic systems have been shown to extend the viability of tissue and can improve physiological modelling of cell, tissue and 3 dimensional or reconstructed tissue systems. Fluidic culture systems help to create a more realistic simulated physiological environment in which to culture cells or tissue and therefore are attractive for the purposes of creating cells and/or tissues to be used in surgical and other applications, but also to simulate in vivo conditions for the purposes of in vitro cell and ex vivo tissue testing.

Known fluidic cell culture systems are typically such that the cells may be exposed to a fluid environment. That fluidic environment may comprise a gaseous or liquid environment. Supply of appropriate fluid culture medium can help to improve overall cell and tissue viability. A number of mechanisms for creating advanced fluidics for tissue culture applications are known, yet implementations are such that full utility of fluidic culture systems has not yet been practically achieved.

By way of example, some issues with known fluidic culture systems are described: fluidic devices typically require movement of fluid within the device to sustain cell viability or to replicate in vivo conditions. Cells being cultured in cell culture devices are typically relatively small samples, sustained in a well of a culture plate. Providing fluidics to a plurality of such small samples may involve significant interconnections external to a device and use fluid lines such as tubing and piping coupled with a network of appropriate valves and fluid pumps. The use of tubing and valves can present some disadvantages. Typically significant lengths of tubing can be required to connect a source of culture medium to each well of a cell culture device and, accordingly, the tubing may itself contain a large volume of unused cell culture medium which is not actively used for the purposes of sustaining a cell sample. Furthermore, a large quantity of tubing may be required to ensure clean supply of cell culture medium to a plurality of samples within a culture device. In other words, the necessary use of tubing containing pumpable cell culture media can prevent effective use of fluidic culture systems to perform large scale testing of biological samples.

Many fluidic cell culture arrangements will require that culture medium is exchanged or renewed in order to maintain biological sample viability. As a result, devices may use peristaltic pumps, requiring extensive tubing as described above. Alternatives for fluid exchange or refresh include use of centrifugal force or some form of physical tipping or movement of an entire unit which can disrupt biological samples under study. Gross physical movement can prevent integration of sensors for biological monitoring and study of samples and can limit the extent to which the fluid or gas flow can be accurately controlled within the cell culture device to mimic physiological conditions which may be experienced by a biological sample in vivo.

Typical organ and skin-on-chip systems are typically enclosed, preventing topical access to skin surface, or suitable access to collect cell or tissue samples for other forms of analysis such as genomic or proteomic testing.

Fluidic systems are typically housed in a CO₂ incubator (for example, at 5-10% CO₂) at a single consistent temperature (for example, at 37 degrees centigrade) in order to mimic in vivo conditions. In some instances, air may be supplied to the apical surface of a tissue/cell sample, but controlled differences in temperature may be difficult to achieve. Typical fluidic systems are not configurable to supply variable gas compositions to, for example, both the apical and basal side of a biological tissue sample. By way of example, fluidic systems may not currently support liquid phase environments having high (for example, 5%) CO₂, low oxygen (for example, below 20%) and allowing for controlled, intermittent pulses of ozone and/or nitric oxide.

Aspects and embodiments may provide cell culture devices and methods which may mitigate some of the issues associated with known fluidic cell culture approaches.

### SUMMARY

One aspect provides a biological sample culture unit according to claim 1.

According to a second aspect, there is provided a biological sample culture module comprising at least two biological sample culture units according to the first aspect.

The module may comprise a gas distribution member or manifold configured to couple to the gas port of each biological culture unit and a gas source.

According to a third aspect, there is provided a biological sample culture system comprising a biological sample culture unit according to the first aspect and/or biological sample culture module according to the second aspect.

Further aspects provide a method of providing a biological sample culture unit according to the first aspect and/or a biological sample culture module according to the second aspect and/or a biological sample culture system or culture apparatus according to the third aspect.

A fourth aspect provides a method of culturing a biological sample comprising: locating the biological sample and a culture medium within a first chamber of a biological culture unit according to the first aspect; providing a reservoir of culture medium and a gas within a second chamber of a biological culture unit according to the first aspect, inducing a pressure difference between the second chamber and the first chamber to facilitate a movement of fluid from the second chamber to the first chamber to circulate cell culture medium from the second chamber to the first.

Aspects recognise that it is possible to provide a biological sample culture unit in which the culturing environment is highly controllable, including the fluidic conditions to which a biological sample is exposed. It is recognised that some cell types, for example, embryonic stem cells, can be particularly sensitive to culture environments. Furthermore, some tissue and cell systems, particularly the skin, use environmental cues to activate changes in biological responses. By way of example, when skin is wounded a sudden change in exposure to high oxygen and low carbon dioxide in subcutaneous layers activates wound healing processes. If exposed to typical culture conditions, in vitro human skin cells forming tissue have been shown to activate the wound healing response, that is to say, known cell culture arrangements for skin cells are not highly controlled enough to provide a realistic “static” imitation of likely physiological conditions. It has, for example, been found that skin or skin cells in typical culture share approximately 30% gene transcription profiles with stress responses akin to hyperproliferation, wound healing and psoriasis. In other words, known fluidic cell culture techniques cannot support successful longer term study of some cells in vitro.

Aspects recognise that it is possible to create a biological cell culture device or unit within which a biological sample can be cultured such that an environment surrounding the biological sample is highly controllable. In particular, a device according to described aspects can allow fluids, for example, liquids (cell culture media and similar) and gases, required to keep the biological sample viable and functional as if in vivo, to be exchanged without exposure of the biological sample to uncontrolled parameters resulting from an external surrounding environment. Arrangements in accordance with aspects may therefore simplify cell culture arrangements, for example, by negating a need to provide complex tissue culture incubators having additional internal doors and chambers to reduce flux in a culture environment; whilst also providing an environment in which longer term sample viability, maintaining a sample as if in vivo, can be achieved.

Aspects provide biological sample culture methods, supportable by devices in accordance with other aspects. The biological sample culture methods of aspects may support various testing methods and modalities requiring use of cultured biological samples.

According to a further aspect, there is provided a fluidic device comprising a first chamber configured to accommodate a fluid, the first chamber being coupled to a fluid pressure regulation chamber via a restricted passage; a second chamber configured to accommodate a reservoir of fluid and a gas, the second chamber comprising a gas port couplable to a gas source; an inlet conduit extending between the second chamber and the first chamber and configured to allow flow of fluid between the second chamber and the first chamber in dependence upon a fluid pressure differential inducible between the second and first chamber.

According to further aspect, there is provided of method of providing such a fluidic device.

According to a further aspect, there is provided a test plate comprising: at least one chamber configured to receive a sample, and at least one probe integrated into the test plate, the probe being configured to measure a parameter associated with the sample locatable within the chamber, wherein the integrated probe is directly coupleable to a contact provided on a printed circuit board placeable adjacent to a surface of the test plate.

According to further aspects, there are provided methods of providing and operating such a test plate.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION OF THE DRAWINGS

Embodiments of the present invention will now be described further, with reference to the accompanying drawings, in which:

FIG. 1 illustrates schematically in cross section some main components of one possible arrangement.

FIG. 2A illustrates in cross-section the main components relating to circulation of fluid within a two-unit module according to an arrangement;

FIG. 2B illustrates schematically in cross-section the main components relating to fluid servicing within a two-unit module according to an arrangement;

FIG. 3A is an isometric representation of some of the main components of a multi-unit module according to one arrangement;

FIG. 3B is an exploded isometric projection of the main components of a multi-unit module such as the one shown in FIG. 3A;

FIG. 3C illustrates a gas channel for use within a multi-unit module such as that shown in FIGS. 3A and 3B;

FIG. 4 is a system diagram which illustrates schematically main components of apparatus which may support use of a unit or module such as those shown in FIGS. 1 to 3;

FIG. 5 illustrates schematically in cross section an alternative embodiment of some main components of one possible arrangement relating to circulation of fluid within a multi-unit module; and

FIGS. 6A and 6B are isometric representations of some of the main components of a multi-unit module according to an alternative arrangement such as the one shown in FIG. 5.

### DETAILED DESCRIPTION OF THE DRAWINGS

Although illustrative arrangements have been disclosed in detail herein, with reference to the accompanying drawings, it is understood that the invention is not limited to the precise implementation described and that various changes and modifications can be effected therein by one skilled in the art without departing from the scope of the invention as defined by the appended claims and their equivalents.

### Biological Background

Various devices exist to facilitate in vitro and ex vivo study of biological samples. Understanding the operation of, for example, small organisms, cells, cell populations and tissues can facilitate further research. For example, in the case of many cells or tissues, the study of stimulus of such cells or cell populations may provide information regarding likely response of those same cell populations or tissues when located in vivo. It will be appreciated that the extent to which a biological sample, such as cells, cell populations or tissues, can be exposed to culture conditions analogous to likely physiological conditions which may be experienced in vivo may impact upon the reliability of information obtained from those in vitro and ex vivo biological samples.

It is recognised that in order to keep cells, cell populations or tissue forming a biological sample, alive and healthy, replication of suitable culture conditions, mimicking those which may be experienced in vivo, may be advantageous. In this respect, fluidic culture systems offer many benefits to cell and tissue culture. They have been shown to extend the viability of the tissue and to improve physiological modelling of cell populations and tissue systems. Fluidic systems are typically such that a culture medium is replaced or updated. That replacement or refresh may occur as a result of direct pumping of the culture medium or by physical removal of a first culture medium before it is replaced by refreshed culture media.

Cell culture systems, including fluidic and microfluidic cell culture systems, may rely upon placement within a closed housing which assists with provision of some control of an environment surrounding the biological sample under study. Some cell culture systems, for example, rely on carbon dioxide incubators to enable pH control and to mimic likely physiological conditions.

By way of particular example, some cells or tissues of interest, for example skin, ocular and lung cells and tissues, are such that culture systems have been provided to create a biphasic culture environment creating a form of air-liquid interface across the cells or tissues under study. At present, most biphasic culture systems, including micro fluidic systems, are created by placement and growth of a biological sample to be studied on a silicone gel, tubing, or plastic material permeable to gases. Known biphasic arrangements may not be configurable to provide a cell or tissue culture environment controllable such that a biological sample is exposed to realistic likely physiological conditions. Access to biological samples within such systems can be challenging. For example, cells in such systems may not be collected at the end of an experiment for invasive forms of analysis such as RNASeq for gene expression profiles.

It will be appreciated that other cells and tissues forming a biological sample may, in vivo, be such that they might form part of an internal organ and thus be entirely immersed in, or surrounded by, a substantially liquid environment.

In order to mimic a range of possible cell and tissue culture conditions which map to likely physiological environments, some culture arrangements are such that culture is conducted in a humidified 5-10% carbon dioxide incubator held at 37° C. and in which the carbon dioxide level is critical to buffer culture media pH. Reduced oxygen, compared to atmospheric levels, in addition to such 5-10% carbon dioxide gas within an incubator, can improve cellular responses such that they more closely match actual physiology. Internal organ physiological oxygen saturation is between 2-5% oxygen and can be as low as 0.2% oxygen in tumour tissue. Low oxygen culture chambers and incubators provide one mechanism to provide a culture environment which more closely matches actual physiology.

Similarly, it is recognised that a gaseous environment surrounding, for example, a tissue being cultured, or a biological sample, is relevant to the creation of a realistic culture environment. Control of a gaseous cell culture environment may also be problematic since it requires tubing and pumping of appropriate gases into or around a cell culture device. It is also recognised that exposure of cells and tissues to gaseous substances, such as reactive oxygen and nitrogen species including nitric oxide ozone and hydrogen peroxide, can have a profound effect on biological function of cells or tissues and therefore may form an area of interest in relation to study of biological samples. Complex physiological intermittent gas supply, such as nitric oxide, ozone and hydrogen peroxide, which are becoming more significant for assessing health and disease tissue, is almost impossible to model with current fluidic systems since there could be a requirement for more than one gas supply.

Described arrangements may provide devices and methods having mechanisms which are capable of providing controllable and reproducible gaseous and environmental arrangements to a biological sample, for example, biological cells or tissue, being cultured. Described arrangements according to the invention may be capable of providing stable, sustainable, reproducible and accurate control of a fluidic (gaseous and/or liquid) environment to which a biological sample is exposed. Described arrangements according to the invention may be capable of providing an environment which supports a range of biological sample test modalities. Some arrangements may provide automated media servicing, some arrangements may provide automated analytical capabilities. Some arrangements may limit uncontrolled disruption of a culture environment and offer ease of use and advanced utility beyond current culture devices and methods.

Before describing one possible particular implementation in detail, a general overview of some main features of some arrangements is provided.

In one implementation there is provided: a biological sample unit comprising two chambers. The first chamber is configured to accommodate a biological sample and to contain a culture medium and a gas reservoir. The culture medium may completely surround the biological sample or may contact only a portion of the biological sample. The first chamber may contain a culture medium in the form of a liquid. The first chamber may contain a gas. The first chamber may contain both a liquid and a gas. The first chamber may be coupled to a gas pressure regulation chamber. The gas pressure regulation chamber may be arranged to support equalisation of pressure within the first chamber and a second chamber and to substantially insulate the biological sample from exposure to a positive pressure. The gas pressure regulation chamber may be arranged to support equalisation or release of pressure within the first chamber to a surrounding environment. That is to say, there is provided some means for the fluid, be it liquid or gas, within the first chamber to escape from the first chamber. That means for escape may allow venting or escape of liquid or gas from the first chamber at a rate slower than fluid may be added to the chamber, and may assist in minimising excessive evaporation from the first chamber. The device may include a second chamber which is configured to accommodate a reservoir of culture medium and a further gas reservoir. The second chamber also includes a gas port which is configured to couple the further gas reservoir to a source of gas. The gas port enables a user to move a volume gas into, or out of, the second chamber. The gas may be moved as a result of operation of a valve to provide addition or extraction of gas to the gas reservoir in the second chamber. The movement of gas into and/or out of the second chamber may be implemented by provision of a device or set of devices configurable to provide a means to add or extract gas to the second chamber via the gas port. By way of example, the gas source may comprise a source of compressed gas, a gas compressor and/or a vacuum pump.

The biological sample unit also includes an inlet conduit which links the second chamber to the first chamber. The inlet conduit may be configured to allow flow of fluid between the second chamber and first chamber in dependence upon a pressure or volume difference between the gas reservoirs in the second chamber and the first chamber. In other words, when gas is added to the second chamber, any gas pressure/gas volume differential between the first and second chamber is such that the inlet conduit provided between the first and second chamber allows fluid, in the form of the culture medium, to flow from the second chamber to the first chamber and vice versa. According to arrangements, the fluid which is able to flow between the second chamber and the first chamber on exposure to a relevant gas pressure/volume increase, or decrease, is the culture medium. The inlet conduit valve is also configured to allow a flow of fluid from the first chamber, in which a biological sample may be housed, back to the second chamber in dependence upon an appropriate gas pressure/volume difference therebetween. The flow from first to second chamber may occur as a result of pressure equalisation, between the first and second chambers. In particular, if the pressure in the second chamber drops, then culture fluid in the first chamber is able to flow from the first chamber back into the second chamber. In other words, when the second chamber is no longer exposed to a positive pressure, culture medium may be free to move from the first chamber, where it may have been in contact with a biological sample, back to the reservoir of culture media housed within the second chamber.

Provision of a gas pressure regulation chamber coupled to the first chamber provides functionality compared to allowing the first chamber to be substantially sealed or closed, which may lead to a pressure increase in the first chamber, with consequent potential to detrimentally impact a biological sample. Similarly, if the first chamber directly and easily vents to a surrounding environment, a required pressure differential to allow transfer of culture medium between the first and second chambers may be more difficult to achieve. Some arrangements recognise that appropriate location and configuration of the gas pressure regulation chamber and the coupling of the first chamber to the gas pressure regulation chamber can allow for efficient operation of the cell culture unit. By way of example, the gas pressure regulation chamber may assist in ensuring reliable and reproducible operation of the cell culture unit. Furthermore, the configuration of the gas regulation chamber with respect to the first chamber may be such that the outlet is shaped to prevent evaporation of fluid and maintain humidity in the gas reservoir of the first chamber by returning water droplets to the first chamber rather than letting such droplets escape to a surrounding atmosphere.

In the arrangements described in detail, fluid contact with a biological sample may always be maintained, even when some fluid is drained from a first chamber to a second chamber, in order to mitigate chances of negatively impacting biological sample viability.

It will be appreciated that arrangements provide a mechanism for achieving a fluidic cell culture environment. A device according to arrangements is configured to support utilisation of pressurised gas and/or changes in gas volume to control fluid flow under the principles of Boyle's Law. Within the device itself, use of Boyle's Law and pressure differences within substantially sealed/fixed volume chambers, ensures that long lengths of piping or tubing in the form of fluid lines external to the unit are not required to move cell culture media around. The conduits provided may be located substantially within the unit. In other words, the conduits may comprise conduits located such that they are internal to the unit.

It will be understood that cell culture media level, and volume of culture medium transfer between the second and first chambers may be a function of inlet conduit dimension, for example, height, length, location in first and second chambers, cross-section and extent, gas pressure/volume change within a chamber, and cell culture medium volume in the second and first chambers. Appropriate balancing of those parameters can result in accurate and repeatable transfer between the second and first chambers of the cell culture device.

The gas port provided in the second chamber may be dimensioned or located such that gas entering the second chamber from the gas source can directly add to or enter the further gas reservoir without passing through a reservoir of culture medium locatable within the second chamber. The gas port provided in the second chamber may comprise a gas permeable membrane which is arranged to prevent fluid entering the gas port.

It will be appreciated that control of fluidic movement within the device by appropriate provision of appropriate internal conduits and application of pressurised gas enables creation of a system which may support, for example, pulsation of cell culture fluid which can, for example, mimic blood flow or blood pressure as may be experienced by a biological sample in an in vivo environment.

Whilst described in relation to a unit formed from a single set of chambers, it will be appreciated that some implementations are such that any number of units, each comprising a set of chambers, can be serviced using an appropriate gas flow distribution manifold which is configured to couple with the gas port of each unit. Arrangements can provide movement of fluid between chambers of each unit which together form a module. Fluid movement within units sharing a gas flow distribution manifold may occur substantially simultaneously. Such arrangements support movement of the same fluid volume in every unit which may have particular advantages in relation to provision of consistent cell culture to support testing and reproducibility of results.

A multi-unit module is described in more detail below. It will be appreciated that it is possible to provide an arrangement in which a plurality of units are substantially simultaneously operable as a result of a single connection between a gas manifold of the multi-unit module to a gas source, thereby achieving scalability for high throughput analysis as may be desirable in relation to testing of biological samples. In other words, a gas port of each of a plurality of second chambers may be coupled, via an appropriate gas manifold, to a common gas source. Common control may be provided, such that control of gas from the gas source results in control of gas being provided to all of the gas ports within the second chambers of a multi-unit module. Arrangements may be such that they also provide for fluid, in particular, culture medium, isolation between units forming a module and, where multiple modules are provided, for fluid isolation between modules.

Returning to general operation of a single unit device: arrangements provide a unit in which appropriate provision of gas to, or from, the second chamber may achieve a movement of fluid between the second and first chambers. Cell culture media circulation may comprise: using gas pressure to implement movement of culture media from a culture medium reservoir within the second chamber to the first chamber in which the biological sample is housed. Arrangements may support circulation by providing a structure in which culture media can be moved from the first chamber to the second chamber as a result of pressure equalisation and/or gravity. As a result, “circulation” of culture media can be achieved between the first chamber and the second chamber. It is possible to implement a regular switch or periodic pattern of adding gas into the second chamber and then allowing an equalisation of pressure between the first and second chambers in order to substantially constantly circulate the culture media past any biological sample within the first chamber.

It will be appreciated that the gas used within the second chamber to affect movement of culture medium from the second chamber to the first chamber may be a matter of user choice. It is possible, for example, to simply use compressed air to increase the pressure in the second chamber and to use a vacuum pump to extract air and reduce the pressure in the second chamber as required. However, the system is compatible with any form of gas and it is possible to use the properties of the gas pumped into or out of the second chamber to alter or adjust the fluid environment to which a biological sample is exposed as well as to control the flow of culture media from the reservoir into the first chamber. For example, it is known to use carbon dioxide in order to adjust the pH of a culture medium. As a result, use of appropriately controlled concentrations of carbon dioxide within the gas used to control a flow of culture fluid from the reservoir to the first chamber may also control the pH of that culture medium. Similarly, oxygen saturation of a culture medium may be altered if a concentration of oxygen within the gas used for fluid movement is adjustable. It will be appreciated that, subject to appropriate control, a system can be provided which is capable of supporting a configurable gas supply of choice to the second chamber in order to achieve a controllable biological culture environment, for example, to match physiological gas levels to maintain a sample at optimal viability, or to expose a sample to typical environmental stress conditions or to assess the impact of altered gas levels, or other adjustable parameter of the culture media. Some arrangements may provide that different gases can be delivered at controlled, intermittent time points, for example, NO, O₃, H₂O₂ and similar. In other words, the gas port of the second chamber may be coupleable to more than one gas source, and one or more gas source may be applied or added to the second chamber as desired to better mimic biological responses or stressors. Appropriate gas source and control arrangements may provide fine-tuned control of gas delivery in both liquid and gaseous phases to the gas port of the second chamber. Some gas delivery systems envisaged for use with, or as part of, the cell culture unit, comprise one or more fine-tuned gas pressure regulator, so that gas from the one or more sources coupleable to the gas port of the second chamber can be delivered in a very controlled manner, for example, when a solenoid valve opens to release pressurised gas from a source towards the gas port.

Gas flow adaptation may allow, for instance, users to investigate the impact of pollution or gas poisoning by appropriate adjustment of the culture media forming the environment surrounding a biological sample.

Some arrangements may allow for capture of gas exhausts from the device. Those gas exhausts may be captured from any gas exhaust outlet of the device. Those captured gas exhausts may be passed through a scrubber to ensure safe use of the device.

Arrangements may include various components which assist in creation of a reproducible, yet realistic, environment in which to culture a biological sample. For example, some arrangements include a heating element and/or cooling element. The heating element may take the form of a thermally conductive layer integrated between fluidic chambers. Some arrangements may include a cooling element integrated between fluidic chambers. That heating and/or cooling element may be configured, with appropriate sensors and feedback control, to create a temperature-controlled environment for the biological sample. In some arrangements, operation of the device components may be configured to hold a fluid phase surrounding a biological sample housed within the device at, for example, 37° C. In some arrangements, a reservoir of culture media or fluid provided in an additional chamber may be held at a temperature which differs from culture media or fluid provided in the second chamber.

Some arrangements are such that further chambers are provided. By way of example, according to one arrangement, service port and media collection chamber can be provided which can enable automated fresh fluid supply and collection at any time of the day without changing the fluidic phase environment. This can help to negate any need to expose biological tissue within the first chamber to an uncontrolled environment if full service of the culture media is required, and/or if extraction of culture media for analysis is required at a particular time.

Some biological samples require biphasic culture, to adequately simulate an in vivo environment. Biphasic culture arrangements are, for example, relevant to the surface of the skin stratum corneum, lung epithelium and the eye corneum. All of which are typically exposed to environmental air whilst the tissue beneath such surfaces are exposed to tissue fluid and/or blood.

Accordingly, in some arrangements the unit comprises: a biphasic biological culture unit which includes a biological sample holder located within the first chamber such that the holder can accommodate a biological sample in a manner that a first surface of the biological sample is exposed to a liquid culture medium and a second surface of the biological sample is exposed to an environment outside the first chamber.

The biological sample holder may be configured to hold the biological sample such that a plug or seal is formed around the biological sample, thereby preventing flooding of a surface of the biological sample which is exposed to the environment outside the first chamber. Such a seal or plug may also prevent leakage of topically applied substances, for example: a drug; product or formulation placed onto the biological sample surface exposed to the environment outside the first chamber into the first chamber.

By arranging the biological sample within a holder such that one surface is exposable to a culture medium located within the first chamber and another surface is exposed to the environment outside the first chamber, it is possible for arrangements to provide a cell culture unit in which the biological sample can be accessible and that sufficient surface area of a biological sample can be available for topical application of a substance; exposure to a particular external environment, and/or inspection or imaging of the biological sample. In some arrangements, where a multi unit module is provided, multiple first chambers may be provided on a plate. Such a plate may be separated from other plates forming a multi-unit module and biological samples within first chambers can be collected or harvested for further analysis.

The environment outside the first chamber may comprise a gaseous environment. The environment outside the first chamber may comprise a filtered gas environment, such that particulate material is excluded from gas entering the environment around the first chamber. The gas environment may comprise air. The gas environment may be controllable and adjustable. Parameters of an environmental gas phase which may be controlled include, for example, gas composition, gas humidity and gas temperature.

In some arrangements, the biological sample culture unit may further comprise a lid which encloses at least a portion of an environment provided outside the first chamber. The lid may enclose a portion of the first chamber and may be configured to provide a controlled environment outside the first chamber to which the second surface of any biological sample located within the sample holder can be exposed. In some arrangements the lid includes: an inlet coupleable to one or more gas sources, including one or more of, for example, filtered air, oxygen, carbon dioxide, and/or other gas sources to which it may be desirable to expose a biological sample under test. In some arrangements the lid includes: an inlet coupleable to a fan or similar device for creating a flow of gas. In some arrangements, the lid includes: an inlet coupleable to a heating or cooling mechanism for adjusting the temperature of gas entering an environment surrounding the first chamber. In some arrangements the lid includes: an inlet coupleable to a humidifier or dehumidifier configured to adjust the humidity of gas entering an environment surrounding the first chamber. In some arrangements the lid comprises an outlet arranged to allow egress of gas from an environment surrounding the first chamber. The outlet and/or inlet may be configured to control the pressure within the gas phase provided outside the first chamber to which a surface of the biological sample may be exposed. The outlet and/or inlet of the lid may be configured to prevent a pressure build up in the gas phase provided outside the first chamber to which a surface of the biological sample may be exposed.

Arrangements may be such that the gas environment surrounding the first chamber may be independently controlled. In other words, the gas phase may be controlled independently of the liquid phase to which a sample may be exposed in a biphasic culture arrangement. Adjustments made to gas supporting the gas phase are distinct and independent to adjustments which may be made to a gas supporting movement of fluid within the first, second and third chambers of a unit or multi-unit module.

In some arrangements, the lid may include a dispenser in the form of a dispensing device, configured to dispense or introduce a test substance to a biological sample housed within the first chamber.

In some arrangements, at least some portions of the lid may be substantially optically transparent to support imaging of a biological sample arranged within the device. Such imaging may, for example, comprise: optical or other microscopy, Raman spectroscopy or similar.

In some arrangements, the lid may include optical components coupleable to an optical imaging apparatus and configured to support imaging of a biological sample arranged within the device. Such imaging may, for example, comprise: optical or other microscopy, Raman spectroscopy or similar.

It will be appreciated that in some implementations the controlled environment to which the second surface of the biological sample can be exposed may also comprise a liquid environment and therefore the two different surfaces of the biological sample may be exposed to different liquid environments. Those liquid environments may comprise different culture media and the biological sample itself may form part of the barrier between the first chamber and the controlled environment enclosed by a lid to which the sample is exposed.

Some arrangements provide a controlled environment which is a gaseous environment provided substantially outside both the first and the second chamber forming the unit. By enabling creation of an environmental gaseous layer on one side of a biological sample it is possible, for example, to achieve a biphasic air-liquid interface culture arrangement which achieves an optimum culture methodology for some types of organ, for example, skin stratum corneum, lung epithelium, or eye corneum.

In each case (liquid-liquid biphasic or gas-liquid biphasic) the chambers and surrounding environment may each be environmentally sealed. The environmental seal keeps liquid and environmental gas phases separate. It will be appreciated that the biological sample itself comprises part of a barrier between the first chamber and the surrounding controlled environment enclosed by a lid. Some exchange, particularly of gas, may occur through the biological sample, as would be likely in a real physiological environment.

In the particular example of a biological sample comprising a skin sample, use of a biphasic unit such as that described in relation to arrangements, can demonstrate that exposure to atmospheric air whilst maintaining “physiological” carbon dioxide and pH levels in the culture medium can allow for maintenance of full skin barrier function and tissue viability beyond 24 hours.

Some arrangements of a unit include one or more sensors or monitoring devices which can be mounted within the first chamber, the second chamber or further chambers, and/or within the controlled environment. Some of those sensors may be configured to sense or monitor a parameter indicative of a characteristic of the biological sample and/or the culture medium in the first and/or second chambers, and/or the gas or liquid housed within the lid forming part of the controlled environment to which the biological sample is exposed.

In some arrangements, a sensor or appropriate monitoring probe, may be coupleable to a printed circuit board (PCB) or similar locatable immediately adjacent to a wall of the first chamber, the second chamber, third chamber and/or within the controlled environment. Arrangement of a printed circuit board within the device can provide a coupling mechanism between one or more probes or sensors and the printed circuit board with substantially cableless connections within the main body of the device. The printed circuit board may include coupling electrodes accessible outside the main body of the device on which wired connectors may be provided. The electrodes may allow the printed circuit board to be connected to a control unit. In this way, sensors or probes located within the device may provide a signal to a control unit. Provision of integrated sensors can facilitate automated and frequent and/or continuous analysis or monitoring and assessment of, for example, the fluid phases forming a culture environment and/or a parameter characteristic of some function of a biological samples housed within a unit according to arrangements. The cableless PCB connections may be sealed or coated to ensure biological compatibility and/or to protect the PCB and PCB contacts, the PCB may be cleanable, the PCB may be compatible with various sterilisation techniques. In essence, the first chamber in which a biological sample may be housed is such that is generally comprises a test plate including the chamber configured to receive a sample, and at least one probe integrated into the test plate and configured to measure a parameter associated with the sample locatable within the chamber. The integrated probe is substantially sealed within the test plate, but arranged such that a surface of the probe is directly couplable to a contact provided on a printed circuit board placeable adjacent to a surface of the test plate.

By way of example, in one implementation an pair of impedance probes are provided for continuous monitoring of a biological sample. The impedance probes may, for example, provide an indication of tissue barrier function in relation to a biological sample where the sample comprises a skin sample. It will be appreciated that such an arrangement can provide a mechanism to assess, for example skin barrier function in relation to applications to test corrosion and irritation in combination with absorption assays. Use of a unit in accordance with described arrangements can provide a mechanism to offer results which can distinguish between levels of chemical irritants and absorption profiles which match a human response after the biological sample has been in the culture environment provided by a unit for five days. Existing skin culture and tissue culture devices have not provided an arrangement which can allow such ex vivo (in vitro) examination of biological samples of skin. Arrangements are such that a conductive surface or contact of each of a pair of impedance probes is accessible on a surface of a test plate in which the impedance probes are integrally placed or sealed and in which the first chamber is formed. A PCB including a surface contact may be locatable immediately adjacent to the surface on which the conductive surface or contact of sensor probe is located. The connection between sensor or probe and PCB may comprise a surface to surface connection. The surface to surface connection may include a spring contact. The PCB and surface of the test plate may be held together by a fastening or clamping mechanism to aid maintenance of a reliable connection between a probe connector and PCB contact point.

Where a PCB is provided, the PCB may include one or more apertures through which a sample locatable within a first chamber may be accessed. Furthermore, such an aperture may allow access to a sample for the purposes of adding a chemical agent to a biological sample to assess the impact of such an agent on the biological sample.

A unit in accordance with various arrangements, whether including a PCB or not, may be configured such that delivery of a substance, for example, bacteria, microbiome, a chemical agent or similar to a biological sample, without significant disturbance to a culture environment, can be achieved. Where the substance is a chemical agent, it may, for example, comprise a drug, compound, or chemical.

A further arrangement can make use of a further chamber. According to such an arrangement, the second chamber may include an additional inlet and outlet linked to the third chamber. The unit may therefore include a third chamber which is configured to accommodate a source of culture media and a gas. The third chamber can itself comprise a further gas port couplable to a gas pump or gas source. The additional inlet conduit to the second chamber from the third chamber may include a one way valve which is configured to allow a flow of fluid from the third chamber to the second chamber when a pressure difference between the third chamber and the second chamber exceeds a further preselected inlet threshold. The arrangement may be such that the additional outlet conduit linking the second chamber with the third chamber is provided such that the additional outlet conduit includes a one way valve which is configured to only allow a flow of fluid from the second chamber to the third chamber when a pressure difference between the second chamber and the third chamber exceeds a preselected outlet threshold.

It will therefore be understood that a unit according to some arrangements may provide two fluidic movement systems activated by appropriate application of positive (or negative) gas pressure to the chambers provided. As described above, one fluid movement mechanism is provided to take culture medium from the second chamber and move it into contact with a biological sample provided in the first chamber. That circuit provides for culture medium to be removed from the first chamber, returned to the second chamber and for culture media in the second chamber to be recirculated into the first chamber.

Provision of a third chamber allows for support of a second fluid circuit activated by application of, for example, appropriate positive and negative gas pressures. Arrangements may be such that substantially the entire content of the second chamber may be emptied into a portion of the third chamber. Having emptied out the second chamber, it is possible to refill the second chamber from a culture medium reservoir or source provided within a separate portion of the third chamber. The source of culture medium may be forced to move from a portion of the third chamber into the second chamber by appropriate application of a positive gas pressure to the third chamber. It can therefore be understood that particular arrangements provide a mechanism to enable fresh fluid supply to a biological sample and collection of media which has been in contact with the biological sample. That collected media may be subsequently analysed, if appropriate to a given biological sample study.

Whilst a particular arrangement is described in detail in relation to a biological sample held in a biphasic arrangement in the first chamber, it will be readily appreciated that the fluid movements and various chambers described in general can provide for effective creation of a circulating fluidic biological system. An appropriate biological sample may, for example, be cultured or held in the base of the first and/or second chamber and bathed in cell culture medium which is exchanged appropriately as described above. Analysis of such bathed biological samples may occur upon separation of the chambers. Such an arrangement may allow for simultaneous exposure of biological samples to an environment and for analysis of inter-related biological samples exposed to related stimuli. Some arrangements may therefore provide for at least three different biological sample types to be simultaneously cultured under fluidic flow within the same footprint.

Devices according to arrangements, when coupled with an appropriate control unit and active components, may therefore provide for automated maintenance (via circulation) and/or servicing of a culture environment, as required to sustain viability of a biological sample. The automated circulation and/or servicing may be scheduled by a control unit to occur at any time of day. Automatic servicing and maintenance of a culture environment can support maintenance of a static and stable culture environment. Accordingly, a consistent culture environment can be provided to a biological sample for an extended period of time and any biological response which may occur as a result of disruption to the culture environment may be minimised or mitigated.

Furthermore, some arrangements may provide a third chamber which is divided into a plurality of portions. Arrangements may provide for one or more differing culture media to be housed within different portions of a third chamber. The different cell culture media may be selected to mimic changes in a culture environment as may result from changes in an environment surrounding a biological sample as a result of a circadian rhythm within a living organism. Cell culture media provided within the chambers may be arranged or provided such that it is possible to supply cell culture media including immune cells or similar active cells within culture media into contact with a biological sample.

Although largely described in relation to a single unit thus far, it will be appreciated that arrangements may be particularly useful when a biological culture module comprising a plurality of such units is provided. A module may include at least two, but likely a plurality of, biological culture units as described above. A biological sample culture module according to arrangements may include a gas distribution member which is configured to couple to the gas port of each biological culture unit and the gas pump. That is to say, that the unit may include a gas distribution manifold. Use of a gas distribution member or manifold allows for common control of a source of gas to be used to effect movement of culture medium within all units forming a module. It will be appreciated that the units maintain their distinct nature and therefore cross contamination of fluid between units is avoided. Accordingly, it is possible to provide a large number of controlled culture environments within a module, thus supporting mass testing of biological samples. Similarly, the module may include a further gas distribution member or manifold configured to couple to the further gas port provided in the third chamber.

It will be appreciated that, within tolerances, if a substantially similar volume of fluid is provided within each chamber of each unit within a module, application of a shared positive pressure to the gas ports via a gas channel/manifold allows a substantially uniform movement of fluid across all units. Such an arrangement supports mass testing of biological samples exposed to a substantially identical culture environment and fluid flow within a module.

Having provided a general overview of some features associated with a unit or multi-unit module, particular aspects of some implementations of a unit, module, apparatus including a unit or module and methods of using the unit, module, or apparatus are now noted in more detail.

Arrangements generally are such that all utilise a source of pressurised gas to control fluid flow within a cell culture unit. Utilisation of pressurised gas to control fluid flow within a cell culture unit has particular application in relation to a module or multi-well system since all fluid flow within the units can be activated from a single gas service point using appropriate gas distribution manifolds. Multi-well arrangements are implemented such that equal volumes of fluid flow, circulation, servicing and collection can be achieved across all units within a module based on the principles of Boyle's Law.

Implementations may be devised such that minimal external fluid lines are required to effect movement of fluid within the chambers of a device. The system achieves fluid flow whilst not directly pumping the culture media. Arrangements enable fluid flow in a manner which does not require peristaltic pumps, use of centrifugal force, magnetic force, surface acoustic waves, optoelectrowetting mechanical actuation and/or microfluidic resistive pulse sensing (MRPS). The system does not require moving parts within the unit, module or apparatus to achieve a flow of fluid within the wells.

Arrangements described may use pressure specific valves, and/or micro filter materials which are gas permeable but prevent transmission of liquid and particulate matter such as dust.

A unit or module in accordance with arrangements can be such that any gas may be used to move the liquid phase (in the form of culture media) between chambers. The gas parameter of significance to ensure operation of the devices as intended is that of applied gas pressure. The gas, of whichever sort, applied to the device may provide for adjustment of the culture media (liquid phase) in order that the liquid phase more closely represents tissue gas physiology and recreates an appropriate environment for a biological sample within the first chamber. For example, use of carbon dioxide as part of the gas being applied to effect fluid flow can allow for adjustment of the pH of the culture media.

Arrangements may be such that in order to create a realistic or appropriate tissue environment an optional air or gaseous environment phase may be provided. The gaseous phase may be provided outside the first chamber and may, for example, take the form of sterile filtered air from atmosphere or from a pressurised gas container.

The liquid phase chambers, namely chambers 1, 2 and 3 may be environmentally sealed and distinct from the environmental air or gaseous phase. The biological sample itself may form part of the seal between the liquid phase and air or gaseous environmental phases created by the unit in use.

In order that an appropriate culture environment is provided to a biological sample, various environmental controls may be provided. For example, a heating mechanism and/or cooling mechanism may be provided to help provide temperature regulation to a unit or module. In other words, chamber, cell culture media and biological sample heating and/or cooling can be provided to provide a substantially consistent or controlled temperature environment to a biological sample. In a module, a common heated plate may be utilised across distinct units. Similarly, some arrangements may be provided as such that a single PCB can be used to support one or more sensors or probes arranged to monitor operation of units in use and/or one or more characteristics of a biological sample provided within each unit of a module.

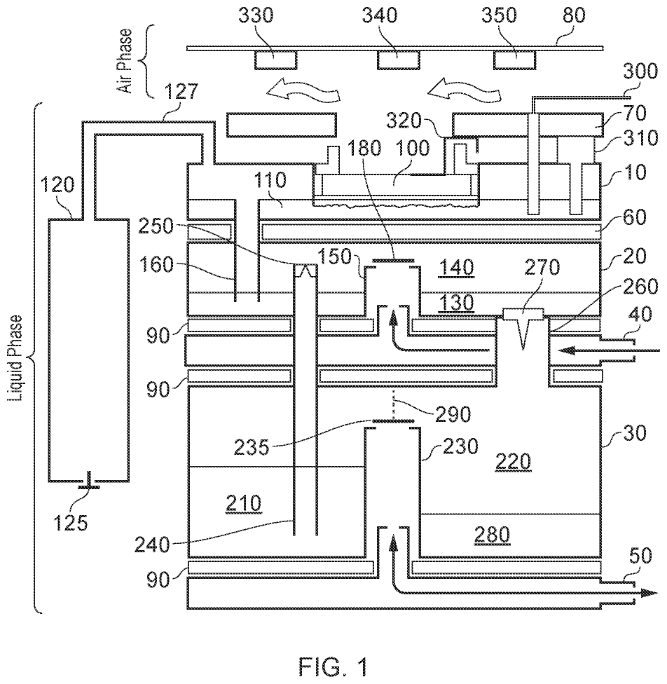

FIG. 1 illustrates schematically in cross section some main components of one possible arrangement. FIG. 1 shows a biological culture unit 1. The biological culture unit 1 shown in FIG. 1 comprises a multilayer stack of components. The arrangement shown in FIG. 1 comprises a first chamber 10 a second chamber 20 and a third chamber 30. The multilayer unit shown in FIG. 1 also comprises a gas channel 40, a further gas channel 50, a heating plate 60, a sensor PCB 70, and a housing or lid 80. A biological sample 100 is locatable within the first chamber and, in the example shown, one surface of the biological sample 100 is exposed to a culture medium 110 and another surface of the biological sample 100 is exposed to a gaseous environment enclosed by lid 80 located above the biological sample 100. Between some fluid-capable layers of the multilayer stack of components sealing gaskets 90 are provided.

The first chamber is configured to accommodate a biological sample 100 and culture medium 110. The first chamber is coupled to a gas pressure regulation chamber 120 which, in the arrangement shown vents the first chamber 10 to a surrounding environment via a small hole located in the region of a sealing plug 125. First chamber 10 can vent gas to allow for zero pressure or item 125 can be in place to block the hole and allow a small regulated pressure build up within item first chamber 10. This is achieved and regulated by the volume of gas pressure regulation chamber 120. In other words, the provision of the sealing plug allows for two configurations—1. Zero pressure build up in first chamber (no plug item 125). 2. Regulated pressure build up in chamber 10 (plug item 125 in place).

The first chamber is configured to house a pool or reservoir of culture medium, and a volume or reservoir of gas. The first chamber and the gas pressure regulation chamber 125 are in fluid communication via a narrow diameter exhaust path 127. The exhaust path 127 acts to restrict, but not prevent, a flow of gas from the first chamber to the pressure regulation chamber and vice versa.

The second chamber 20 is configured to accommodate a reservoir of culture medium 130 and includes a space or volume which accommodates a further reservoir of gas 140. The second chamber 20 includes a gas inlet 150 couplable to a gas source (not shown in FIG. 1). An inlet conduit 160 links the second chamber 20 with the first chamber 10. The inlet conduit extends from the second chamber 20 into the first chamber 10. The inlet conduit 160 is configured to allow a flow of fluid from the second chamber to the first chamber and vice versa. The inlet conduit is configured such that culture fluid 130 located in the second chamber can enter towards one end of the inlet conduit 160. The inlet conduit extends into the first chamber. The inlet conduit has a preselected height or extent to which it protrudes into the first chamber from the base of the first chamber. Under conditions as described further below, culture medium can flow from second chamber 20 into the first chamber 10 through the inlet conduit 160.

In use, a source of compressed gas (not shown in FIG. 1) is controlled by an appropriate valve, for example, a solenoid valve, and as desired to control fluid movement within the unit can supply gas, in this case, air, through gas channel 40 into the second chamber 20 via gas port 150. The gas port 150 is provided with a membrane 180 through which gas may pass but fluid may not. Supply of air into the second chamber via the gas port 150 results, as will be understood via a simple application of Boyle's Law, in the culture medium housed within the second chamber 130 being pushed into inlet conduit 160 towards the first chamber 10. The pressure difference between the second and first chamber induced by a supply of additional gas to the gas volume of the second chamber results in a gas pressure increase in the second chamber and fluid flow from the second chamber 20 into the first chamber 10. The addition of fluid to the first chamber does not result in a consequent increase in gas pressure within the first chamber (provided the sealing plug 125 of the gas pressure regulation chamber is not in place) since the gas located in the first chamber may leave the first chamber via the restricted exhaust path and, as a result, the biological sample is largely insulated from exposure to a positive pressure increase. If the addition of gas to the second chamber is maintained or released, the system comprising second chamber, first chamber and gas regulation chamber operates to return to equilibrium and it will be appreciated that the return to pressure equilibrium between chambers can result in a flow of fluid from the first chamber into the second chamber via the inlet conduit. The volume of culture medium 110 in the first chamber and the culture media reservoir 130 provided within the second chamber, the diameter of the inlet conduit, the extent of the inlet conduit which extends into the first chamber and the applied addition of gas to the second chamber are selected such that sufficient transfer of spent culture medium and new culture medium is achieved to maintain viability of the biological sample 100.

It will be appreciated that dimensions of various components can be selected to support effective operation of the device in use. For example, the extent to which the conduit extends into first chamber 10 may be selected to support maintenance of an appropriate level of culture medium 110 within the first chamber. As shown in FIG. 1 biological sample 100 is not completely immersed within culture medium 110 and instead only one surface of biological sample 110 is exposed to direct contact with culture medium 110. Similarly, the extent to which gas port 150 extends into the second chamber may be selected such that a level of fluid 130 within the first and second chambers are appropriately maintained and that the conduit 160 has an intake opening located within fluid rather than gas locatable within the second chamber.

In the arrangement shown in FIG. 1 there is also provided a third chamber 30. The port 180 provided in the second chamber 20 which includes the gas, but not fluid, permeable membrane acts as a gas path from the second chamber as required. In the arrangement shown, the second chamber can allow some escape of gas via the gas permeable membrane into gas channel 40.

The third chamber 30 is configured to accommodate a source of culture medium. In the example shown, fresh fluid 210 is located in the third chamber. Remaining space 220 within the third chamber is filled with a gas. The third chamber 30 includes a gas port 230 which is couplable, via gas channel 50, to a gas pump (not shown in FIG. 1). The gas port 230 includes a port membrane 235. An inlet conduit 240 linking the third chamber with the second chamber is provided. The inlet conduit 240 includes a one way valve 250. The one way valve 250 is configured to allow a flow of fluid from the reservoir 210 in the third chamber into the second chamber via the conduit when a pressure difference between the third chamber 30 and the second chamber 20 exceeds a selected further inlet threshold value. The second chamber 20 includes an outlet conduit 260 which links the second chamber 20 with the third chamber 30. The outlet conduit includes a one way valve 270 which is configured to allow a flow of fluid, for example fluid 130, from the second chamber 20 into the third chamber 30 when a pressure difference between the second chamber and the third chamber exceeds a different further outlet threshold value. It can be seen in FIG. 1 that chamber 30 is split into two portions and that fresh fluid 210 is kept separate from fluid 280 which is drained from the second chamber. The two portions of the third chamber are, in the arrangement shown schematically in FIG. 1, separated by a fluid isolation barrier 290 which keeps the fluids separate but is gas permeable, such that positive and negative gas pressures applied to the third chamber by a gas pump are applied across the whole third chamber.

Operation of fluid flow between the third chamber and second chamber can be affected by appropriate use of a valve coupled to a gas source, gas compressor or a vacuum pump connected to gas channel 50. The mechanism by which “servicing” fluid flow occurs between the third chamber and the second chamber is largely analogous to that described in more detail above for moving fluid between the second and first chambers. In short, pushing gas into or extracting gas from, the third chamber 30 adjusts the pressure within the third chamber causing a consequent movement of fluid from the third chamber into the second chamber and vice versa. Unlike “circulatory” fluid movement between the second and first chambers, the implementation shown in FIG. 1 is such that no specific gas pressure regulation chamber is coupled to the second chamber. Instead, the gas channel 40 can be utilised to regulate pressure within the second chamber.

It will be appreciated that gas channels 50 and 40 may be couplable to the same gas source via different valve and control arrangements. Whether the gas is supplied or held in or to gas channel 40 or 50 can be controlled by appropriate use of valves within a gas circuit (not shown in FIG. 1) of a system controlled by an apparatus control unit (not shown in FIG. 1).

The arrangement shown in FIG. 1 includes a heating plate 60 located between the first chamber and second chamber in order to provide biological sample 100 with a temperature controlled environment.

The biological sample 100 in FIG. 1 is exposed to a biphasic culture arrangement. One of its surfaces is in contact with culture medium 110 within the first chamber 10 whilst the opposing surface is exposed to an air phase controlled environment enclosed by a lid 80 provided above the first chamber 10.

The arrangement shown in FIG. 1 includes a temperature probe 300 which extends into the first chamber, fluid impedance probes 310, 320 which extend into the first chamber. Each of those probes can be couplable to a printed circuit board 70 which itself is couplable to a control unit (not shown in FIG. 1), such that monitoring and feedback control of a culture environment, air phase and a biological sample supported by the device shown in FIG. 1 can be implemented.

FIG. 1 shows a unit in which return of fluid from the first chamber to the second chamber is achieved via equalisation of pressure between the chambers and gravity. The Extent to which that return happens, and speed of return may depend upon the sealing plug. If the sealing plug is such that in imperfect seal to a surrounding environment is provided, then the pressure between the first chamber, second chamber and gas pressure regulation chamber all equalise to ambient pressure.

The arrangement of FIG. 1 is such that a single internal path/conduit is provided between the first chamber located in the upper well plate and the second chamber located in the lower well plate is provided for fluid circulation between the second and first chambers.

In the arrangement of FIG. 1, the first chamber vents via a ‘dog-legged’ exhaust path. The shape of the exhaust path has been selected to traps humidity and prevents evaporation of fluid allowing for maintenance of a stable biological sample culture environment.

The arrangement of FIG. 1 is such that a gas pressure regulation chamber is included. The regulation chamber is configured to regulate the gas pressure under the biological sample. If the sealing plug is removed from the chamber then a zero pressure is achieved under the biological sample.

In the arrangement of FIG. 1, the regulation chamber also acts as humidity trap due to its positional level below the Upper Well (first chamber). The volume of the regulation chamber has been selected to prevent mixing of atmospheric gas with internal gas under the biological sample when the system is momentarily not under positive pressure.

### Well Unit Concept

In the example arrangement shown in FIG. 1, a biphasic culture arrangement is provided. An air phase (which may, in some arrangements, comprise a gas other than air) is provided above sensor PCB 70. A liquid phase is provided beneath sensor PCB 70. The top of biological sample 100 is exposed to the air phase and the bottom surface is within the liquid phase.

A seal is maintained between the liquid phase and the air phase. The biological sample itself 100 forms part of that seal. Otherwise, the seal is maintained between the top and bottom surface of the biological sample 100 by appropriate component arrangement.

### Liquid Phase

In the arrangement shown in FIG. 1, everything beneath the sensor printed circuit board 70 is referred to as the “liquid phase”. The fluidics in the liquid phase, including the culture medium 110, 130 and 210 and sensors 300, 310 and 320 are configured to provide controllable and reproducible conditions for biological culture and testing of biological sample 100.

The liquid phase sensors 300, 310 and 320 can be connected to the sensor PCB 70 without a need for wires and specific connections. The sensors are mated to the bottom surface of sensor PCB 70 via surface to surface to contact thereby allowing for ease of installation and removal of the sensor PCB as required.

### Air Phase

Within the air phase there are also provided various sensors. In the example shown in FIG. 1 there is provided: a humidity sensor 330, a carbon dioxide sensor 340 and a temperature sensor 350. Within the air phase there can be forced air movement via, for example, use of a fan or application of compressed or pressurised gas or air (not shown in FIG. 1). Regulation of the air phase to provide controlled conditions to a biological sample 100, is achieved via provision of the sensors which provide signals to a control unit (not shown in FIG. 1) which provides for monitoring and feedback control of the air phase.

It will be appreciated that signals from all sensors (300, 310, 320, 330, 340, 350) can be passed to an appropriate control unit (not shown in FIG. 1) which is configured to implement feedback control and appropriate adjustment of operational characteristics of the device in use to secure a stable culture environment.

### Temperature Regulation

Heat plate 60 is configured to provide or maintain a controlled temperature to culture medium within the first chamber. A uniform temperature gradient across the biological sample may be achieved by use of a heating plate comprising a heat mat upon a thermally conductive substrate.

The temperature of the culture environment surrounding the liquid phase of the biological sample can be regulated via appropriate use of a signal from one or more temperature probes provided in the liquid phase. A temperature probe may be performed directly via a temperature probe inserted directly into the culture media. Alternatively, a temperature probe 300 may comprise a metal probe having known thermal properties. Use of such a temperature probe can help to eliminate the need for any difficult to handle wires or permanent connections. The temperature probe 300 may provide an upper surface which supports surface to surface thermal meeting between the upper surface of the temperature probe and a conductive portion of the sensor PCB 70. Such an arrangement supports thermal mating which allows for ease of assembly and operation. A temperature sensor on the sensor PCB may be configured to measure the temperature at the PCB junction and then software provided at the electronics control unit (not shown in FIG. 1) is configured to factor in an offset temperature which will remain constant when at a steady state.

### Module

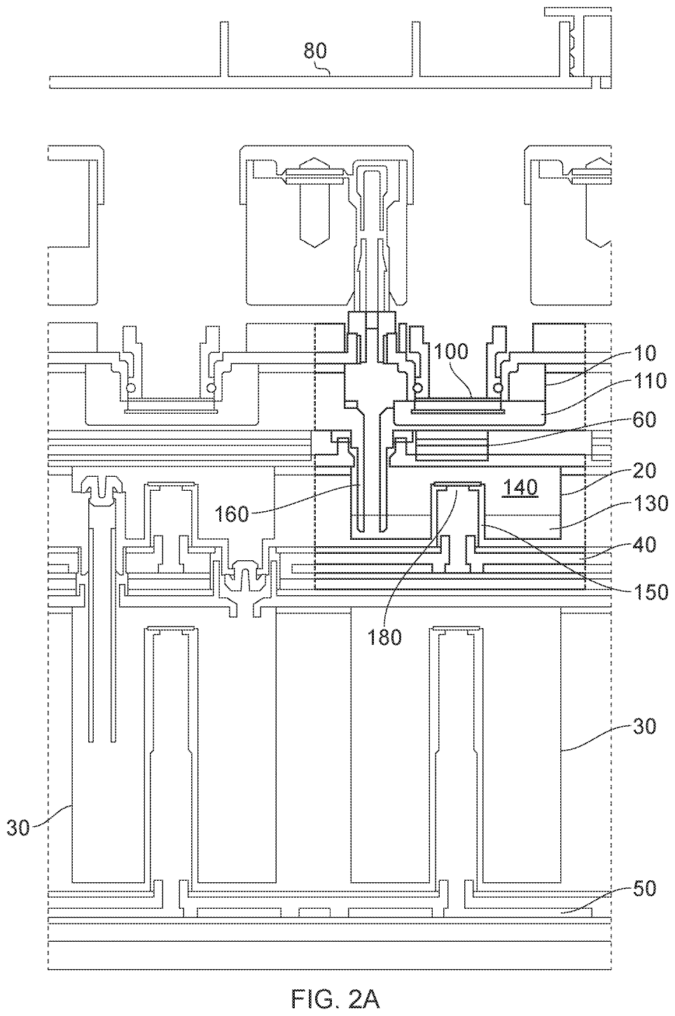

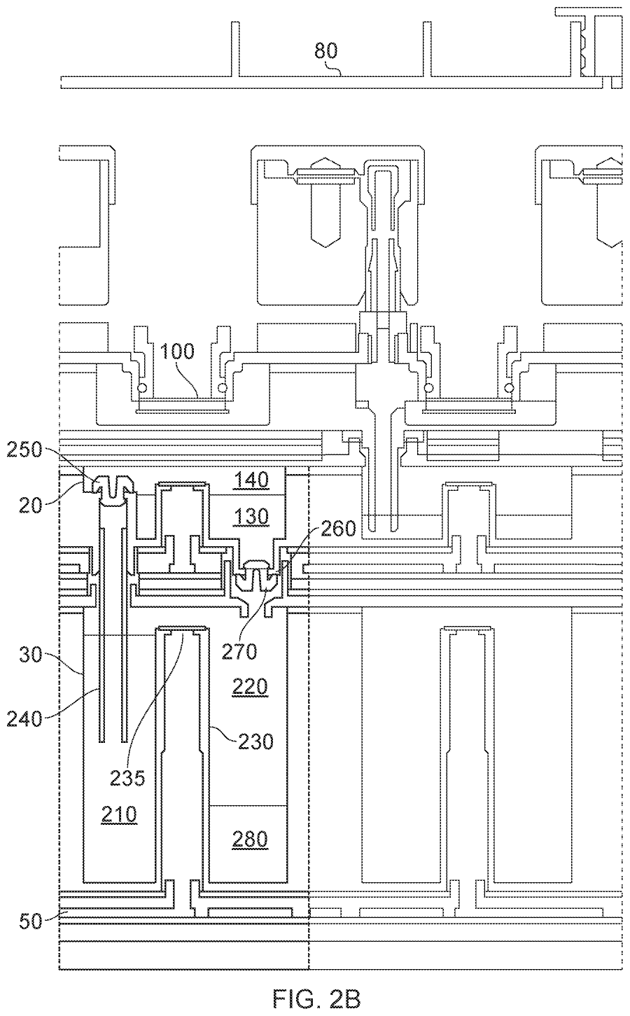

FIG. 2A illustrates in cross-section main components of a two-unit module. The components involved in fluid circulation between a first and second chamber are highlighted. FIG. 2B illustrates schematically in cross-section main components of a two-unit module. Main components relevant to fluid servicing between a second and a third chamber are highlighted. Where appropriate, the same reference numerals have been used throughout the Figures to denote the same components. The gas pressure regulation chamber shown in FIG. 1 is omitted from FIG. 2 for the purposes of clarity. It will be appreciated that a common gas pressure regulation chamber may be provided which is common to all first chambers within a module comprising more than one unit.

### FIG. 2A: Fluid Circulation

In order to circulate fluid in contact with a biological sample 100 located within first chamber 10, fluid circulation steps may be taken:

Step one: gas may be supplied to gas channel 40 and membrane 180 is configured to allow a flow of gas into the second chamber.

Step two: the addition of gas to the second chamber cases an increase in gas pressure in the second chamber, and in order to release that pressure, culture medium fluid is forced to travel up via inlet conduit 160 from the second chamber into the first chamber. The fluid travels up via the inlet conduit 160 towards (lower pressure) chamber one. In other words, as a result of a pressure difference between chambers, fluid feed between a reservoir housed in the second chamber to a culture environment within the first chamber is achieved. The addition of fluid to chamber one causes a temporary increase in pressure within the first chamber which is released/equalised via the exhaust path to the pressure regulation chamber.

Step three: As pressure in the second chamber equalises, fluid added to the first chamber can return to the second chamber. Fluid in the first chamber drains back into the second chamber under the influence of gravity via the same inlet conduit.

The circulation process of steps 1 to 3 can be continued until required circulation of fluid is achieved.

### FIG. 2B; Fluid Servicing

Movement of fluid between the second chamber 20 and the third chamber 30 is generally referred to as fluid servicing and can occur by means of the following steps:

Step one: gas enters chamber three via gas channel 50.

Step two: Gas flows from the gas channel into chamber three of each unit. The volume enclosed by chamber three is pressurised by the influx of gas. The pressure increase causes fresh culture medium to travel up into the second chamber via inlet conduit 240. In other words, when gas flows into the third chamber, the second chamber is at a lower pressure than the third chamber and therefore fluid feed between the third chamber and second chamber can be achieved.

Step three: negative gas pressure can be applied to the gas port of chamber three of each unit via gas channel 50. The volume enclosed by the third chamber is then under negative pressure which allows used fluid within the second chamber to drain, via outlet conduit 260, into the third chamber from the second chamber. In other words, application of negative pressure to the gas channel 50 causes a fluid drain from the second chamber into the third chamber. Arrangements may provide a split, subdivided or multi-portion third chamber such that fresh media for servicing and collected media from the circulation chamber can be kept separate and do not mix.

### Multi-Unit Module

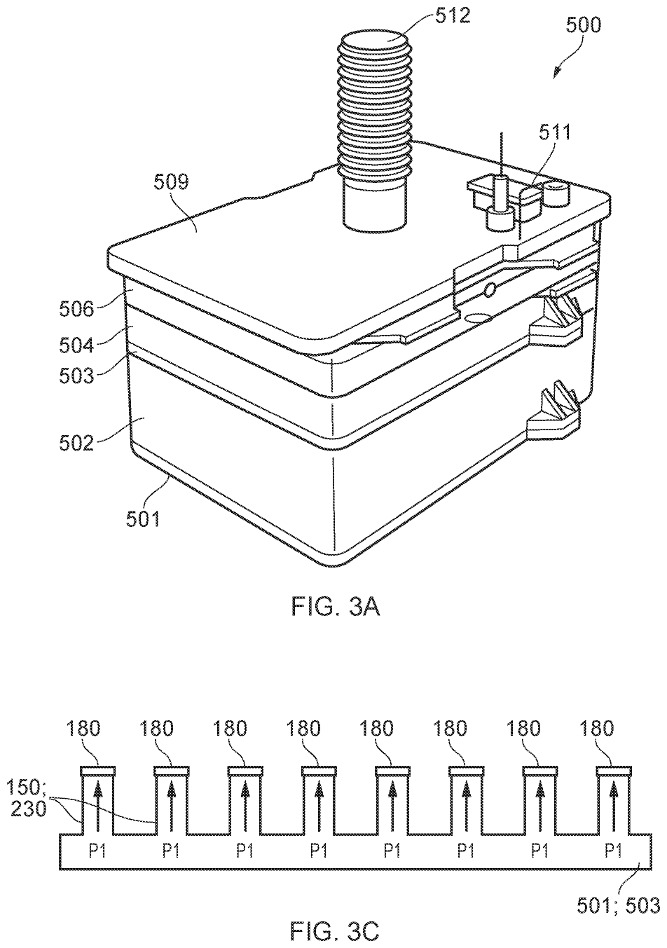

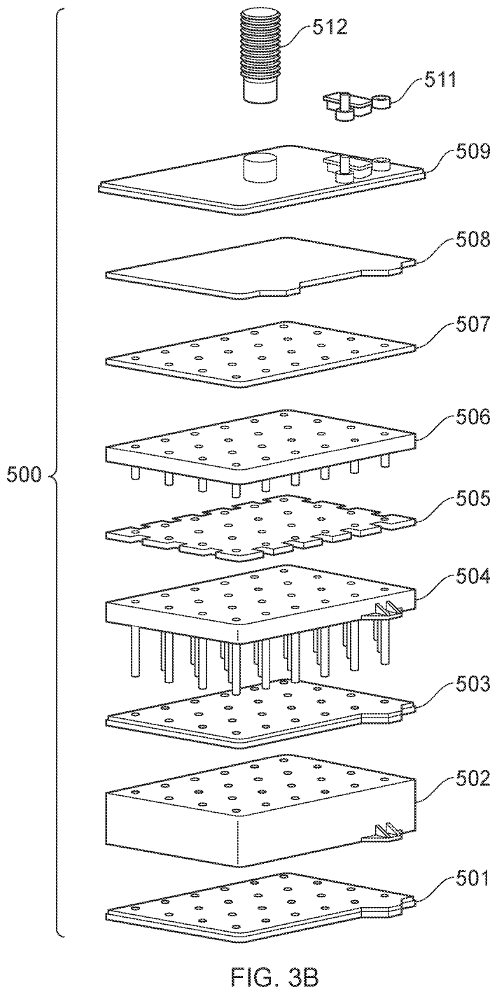

FIG. 3A is an isometric representation of some of the main components of a multi-unit module according to one arrangement. FIG. 3B is an exploded isometric projection of the main components of a multi-unit module such as the one shown in FIG. 3A. The gas pressure regulation chamber shown in FIG. 1 is omitted from FIG. 3 for the purposes of clarity. It will be appreciated that a common gas pressure regulation chamber may be provided which is common to all first chambers within a module comprising more than one unit.

The module 500 illustrated in FIG. 3A comprises: an upper well plate 506 which comprises a plurality of first chambers as shown in more detail in FIG. 1 and FIG. 2. The module 500 also comprises a lower well plate 504 which comprises a plurality of second chambers as described in relation to FIGS. 1 and 2; and a service tank 502 comprising a plurality of third chambers as described in relation to FIGS. 1 and 2. The module 500 also comprises: gas channel 501, gas channel 503, heat plate 505 (not visible in FIG. 3A), a gas exhaust plate 507, a sensor PCB 508, an air phase cover or lid 509, air phase sensors 511 and an air hose 512. The various plates shown in FIGS. 3A and 3B are fastened together to allow the internal chambers of the units forming the multi-unit module to be pressurised in support of the fluidic mechanisms described.

The fastener may, for example, comprise a clamp, or similar. It will be appreciated that in all arrangements (single unit and multi-unit) the chambers are configured within the device to allow for pressurisation and depressurisation.

### Fluidic Concept

As described in relation to the single unit of FIG. 1 and the two unit arrangement of FIG. 2, the multi-unit module 500 supports a culture environment by providing mechanisms within the device which support fluid flow within each unit forming the module. In the arrangements shown in FIGS. 1 to 3 both fluid circulation and fluid servicing are supported, though it will be appreciated that simpler devices in which only circulation, rather than circulation and servicing, of fluid is supported are also possible.

### Fluid Servicing

Fluid servicing is the term used to refer to draining used fluid from the second chambers and replenishing the second chamber of the system with fresh fluid. For this purpose, in the multi-unit module shown in FIG. 3, items 501, 502 and 504 are used. It will be appreciated that typically the service tank 502 comprising a set of third chambers is pre-loaded with servicing fluid. Positive pressure can be used to move fluid from the service tank 502 to the lower well plate 504. Negative pressure is used to reverse the flow and to drain fluid from the lower well plate into the service tank. Gas applied via gas channel 501 or extracted from gas channel 501 is used to create uniform air pressure or vacuum across units forming the multi-unit module.

### Fluid Circulation

Fluid circulation is the term used to describe a flow or exchange of fluid in contact with a biological sample within the first chamber of each of the units forming multi-unit module 500. For this purpose, items 503, 504 and 506 are used. The lower well plate 504 contains the fluid for circulation (which originally came from the service tank 502 during the servicing phase). Positive pressure can be used to move the fluid from the lower well plate 504 to the upper well plate 506 equalisation of gas pressure can be used to reverse the fluid flow and drain fluid from the upper well plate 506 into the lower well plate 504. Gas used to create uniform pressure across units enters the lower well plate via the gas channel 503.

FIG. 3C illustrates a gas channel in the form of a gas manifold for use within a multi-unit module such as that shown in FIGS. 3A and 3B. The air channel shown forms a gas distribution manifold configured to supply the plurality of units forming multi-unit module 500. The gas channel comprises a plurality of gas ports. The gas channels 501 and 503 of the module 500 shown in FIGS. 3A and 3B are configured to supply gas to service tank (comprising third chambers) 502 or lower well plate (comprising second chambers) 504 to enable and facilitate fluid servicing or fluid circulation as appropriate.

Within the service tank 502, lower well plate 504 and upper well plate 506 there are provided individual wells containing fluid and/or gas. All of the wells are isolated from each other and so the fluids are isolated within each plate, and within each unit, formed by a stack of wells in adjacent stacked plates. Arrangements allow for fluid servicing and circulation whilst still maintaining isolation between units. Such isolation prevents cross contamination between units. The isolation is not compromised by use of common gas channels to implement pressurised volumes within each unit, since appropriate arrangement of components allows movement of fluid between chambers of each unit simultaneously but independently. The principle works based on Boyle's Law which maintains a constant pressure within each of the gas channels 501 and 503. When a gas is supplied to each gas channel/manifold, the pressure in the closed-volume gas channel increases. The gas permeable membrane is selected (within tolerance) to allow gas to permeate when exposed to approximately the same gas pressure. As a result, when all of the units are exposed to the necessary increase in pressure, they will substantially simultaneously “open” and allow a flow of gas from the air channel into, for example, the third or second chamber. It will be appreciated that Boyle's Law is summarised by the following equation: P₁V₁=P₂V₂ where P is pressure and V is volume. Once there is sufficient build up of pressure within a gas channel/manifold the gas permeable membrane allows passage of gas substantially simultaneously and provides equal amount of pressure within the chambers (each also having the same volume) for isolated fluid movement within each unit. The reverse is true if negative pressure is applied to enable fluid return.

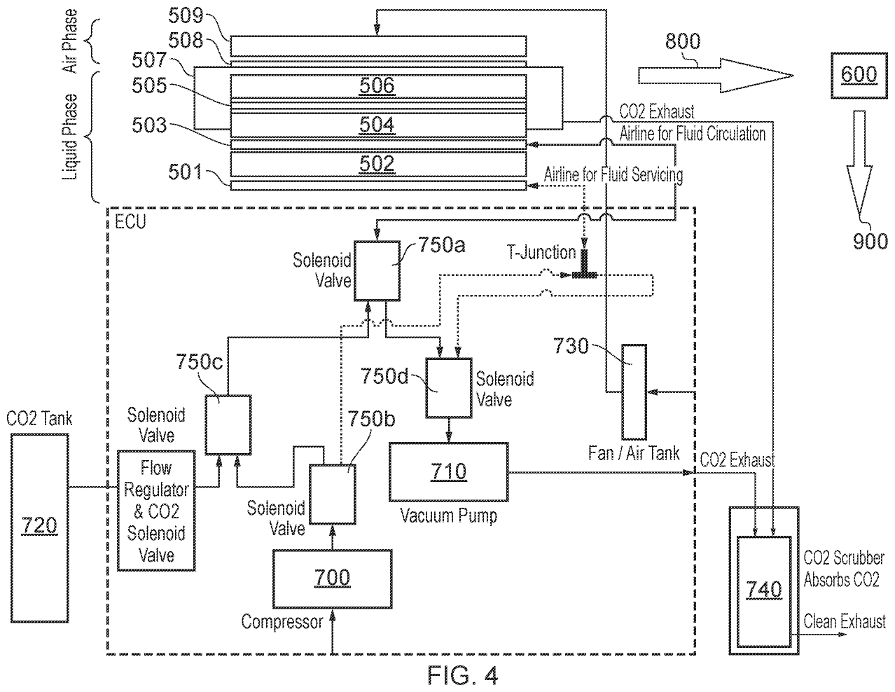

FIG. 4 illustrates schematically interaction of some main operational components of apparatus configured to support use of a unit or module such as those shown in FIGS. 1 to 3.

A representation of main components of a circulation stage of a multi-unit module is shown forming part of the apparatus of FIG. 4. Gas channel plate 503 is arranged beneath a lower well plate 504. A heat plate 505 is interposed between lower well plate 504 and upper well plate 506. A gas exhaust plate 507 is provided above the upper well plate 506. A sensor PCB 508 is located above the upper well plate 506. An air phase cover 509 is located above all the other plates forming the multi-unit module. The cover 509 encloses the other plates forming the multi-unit module and provides a contained volume in which an air phase can be provided to any biological samples within the multi-unit module.

Sensors provided in the air phase, for example, a humidity sensor, carbon dioxide sensor and/or temperature sensor (330, 340, 350 in the arrangement shown in FIG. 1) may be configured provide 800 a signal indicative of the property they are measuring or monitoring to a control unit 600. Those signals may be continuous or periodic. The control unit may be configured to store the values reported, and/or control operation of a apparatus via control signals 900 sent from the control unit 600 to one or more operational elements of a system. Feedback control may be implemented by the control unit in dependence upon the signals received. For example, if the air phase temperature is reported to be too high, the control unit may be coupled to, for example, a cooling element, and instruct appropriate air phase cooling until the signal received by the control unit reports an acceptable temperature has been reached.

The control unit 600 is also couplable to sensors provided to monitor operation of the multi-unit module within the liquid phase. For example, control unit 600 may be configured to implement feedback control based upon signals and information received from the sensors forming part of the multi-unit module to ensure smooth operation of the device and maintenance of a consistent culture environment for biological samples located within units forming module. In particular, control unit 600 may be coupled to one or more sensors or probes and configured to activate one or more components of an apparatus to cause operation of a biological sample culture method as described in more detail below. For example, if the liquid phase temperature is reported to be too low, as a result of signal received from temperature probe 300 the control unit may be coupled to, for example, a heating plate 60; 505, and can instruct appropriate liquid phase heating until the signal received by the control unit from the temperature probe 300 reports an acceptable temperature has been reached.

The apparatus shown schematically generally in FIG. 4 may comprise: a compressor or source of compressed gas 700 configured to provide compressed gas to air channels; a vacuum pump may be provided 710 and configured to provide negative gas pressure/suction to one or more air channel for servicing, for example; carbon dioxide tank 720 configured to provide a flow of carbon dioxide into a gas flow providable to the air channel 503; a fan or source of gas 730 configured to control a flow of air to the air phase; a scrubber 740 configurable to receive a flow of gas from gas exhaust layer 507 and the air phase environment enclosed by air phase cover 509.

Operation of the apparatus may be controlled by the control unit, which is configured to provide appropriate control signals 900 to a plurality of solenoid valves 750 configured to allow control of an air flow to and from air channels. The system may, in some arrangements, include one or more flowmeter regulators (not shown in FIG. 4) configured to send an indication of gas flow to/from gas channels. Flowmeter regulators configured to control gas flow into the chambers of the liquid phase may aid fine control of fluid flow within a unit or multi-unit module. In dependence upon signals received from the control unit 600, a solenoid valve 750a may operate to allow a positive gas flow into the gas channel from compressor 700. That flow, for example, of compressed air, from the compressor 700, to the gas channel, is also controlled by operation of solenoid valves 750b and 750c. As described in relation to FIGS. 1 to 3, application of an appropriate positive gas pressure to the lower well plate causes fluid in the lower well plate (second chambers) to move from the lower well plate into the upper well plate (first chambers). If it is desired to adjust the pH of the culture media being supplied from the lower well plate into the upper well plate, the control unit 600 may operate such that solenoid valve 750c allows a flow of carbon dioxide from carbon dioxide tank 720 to join a flow of compressed gas from the compressor 700 to the gas channel 503. In some arrangements, when compressed gas 720 is used, it does not join with compressed gas from compressor 700. Compressed gas 720 is pre-pressurised and does not rely on 700. Valve 750c shuts the path from 700 to 750a and opens path from the compressed gas source 720 to valve 750a.

If it is desired to apply a negative pressure to a gas channel plate the control unit 600 may cause operation of solenoid valves 750a, 750b, 750c and 750d and activation of vacuum pump 710 such that air flows from the gas channel plate and fluid can be moved between chambers provided on well plates.

Similar control by the control unit 600 can be implemented if it is desired to move air within the air phase. In particular, the control unit 600 may be configured to activate fan 730 or an appropriately provided solenoid to provide a supply of pressurised air or gas to the environment provided between the first chamber and the lid of the device.

Although particular features of arrangements and implementations are described in detail above, it would be apparent to a person skilled in the art that arrangements provide various functions and features.

In particular, within the liquid phase of some arrangements: where are three liquid phase chambers; one pair facilitate fluid circulation and one pair facilitate fluid servicing. Fluid servicing fluid flow provides fresh media delivery and media collection. Fluid flow can be continuous or pulsated. Fluid servicing can be continuous or intermittent. Fluid flow rate and servicing can be controlled by a control unit implementing appropriate software. Parameters of operation can be adjusted according to the needs of a user and/or a particular culture environment to be provided to a biological sample. Media flow can be pulsated, and can be set to a rhythm similar to blood pulsation. Media flow and servicing is controlled by application of pressurised gas. Theoretically any gas can be used to effect fluid flow between chambers of a device, including, for example, air, carbon dioxide, oxygen, nitrogen, ozone, nitric oxide, hydrogen peroxide, and/or argon. The system is designed to provide physiological gas control within a culture media being provided to a biological sample. Gas levels within the culture media may, for example, include 5% carbon dioxide, 2-5% oxygen to mimic an appropriate physiological environment within the culture environment. The system can also be used to provide pulsations of reactive oxygen and nitrogen species, including nitric oxide, ozone and hydrogen peroxide to a biological sample. Reactive oxygen and nitrogen species, for example, are known to influence biological processes, both positively and negatively in healthy and diseased cells.

The system utilises gas pressure to move fluid/media in a unitized system, enabling multi-well fluidic flow to be achieved with minimal use of gas pumps (a circulation only system may, for example, utilise a single source of compressed gas). Fluid flow within a device can be achieved without fluid contamination between units. Arrangements may utilise appropriate mechanisms to facilitate directional fluid flow between chambers in dependence upon changes of pressure. For example, one mechanism may comprise use of valves with differing operating pressure to facilitate direction of fluid flow and achieve either fluid circulation or fluid servicing. Alternative mechanisms utilise conduits between chambers which have appropriately selected dimensions, for example, protrusion extent into each chamber, to facilitate direction of fluid flow and achieve either fluid circulation or fluid servicing.

Components of a device can be dimensioned and located such that valves and/or conduits are situated at positions within chambers of a device which maintain a desired minimum or maximum fluid level within a chamber. For example, valves may be located within the first chamber such that a level of fluid is maintained within the first chamber which ensures an underside of a biological sample within the first chamber is in constant contact with the cell culture fluid. By way of further example, conduits may have heights and protrusions between chambers which are selected such that a level of fluid is maintained within the first chamber which ensures an underside of a biological sample within the first chamber is in constant contact with the cell culture fluid.

Although reference is made to use of valves, it will be appreciated that some valves may be replaced with gas permeable filter material and can be selected to be permeable to gas but not liquid, thus maintaining liquid phase separation, is biologically inert, can be sterilised by autoclaving and can function under the pressures typically used within the sample culture system. By way of example, a filter material which is gas permeable but prevents the passing of liquid and particulate matter is envisaged for use within the third (servicing) chamber to aid separation between fresh and drained fluid reservoirs.

The media servicing chamber (chamber three) may, in some arrangements, comprise two or more separate portions or wells. One well may be configured to supply fresh media, another configured to receive used collected media. Arrangements may be such that fresh media for servicing and used media collected do not mix, enabling chemical analysis of media upon collection. Some arrangements of the system may enable automated media servicing and collection which can be controlled via software by a user.

The media flow chambers may be held at a substantially constant temperature, for example, 37C, by use of an appropriate heating element, for example, a heat mat. The system can be scaled or miniaturised. Volumes of fluid moved in the media circulation, servicing and collection phases can be adjusted by a user by adjusting gas flow with a software package implemented by a control unit.

The multi-unit module shown schematically in FIG. 4 comprises 24 units, but it will be appreciated that any number of wells could be provided and controlled by a centralised gas pump and gas pressure. In the multi-unit module illustrated in FIG. 3, ‘gas channels’ are used as gas distribution manifolds configured to provide a uniform pressure across multiple units within the module.

Operation of all units, modules and apparatus described herein is based upon Boyle's Law. The principles of Boyle's law support a uniform fluid flow, delivery and collection at equal volumes across all wells. In particular, it will be appreciated that the length of a gas path to various units within a module does not alter the volume of liquid movement/flow/collection which is achieved within the various units.

In other words, the principle of Boyle's Law is such that across all units within a module, the pressure applied is uniform. Therefore whatever pressure is applied to one unit will be the same throughout all units. This may be achieved within a device by using ‘air channels’ which use a shared gas volume to apply pressure across all the pressure inlets of each of the units.

Boyle's law states that for an ideal gas at a constant temperature: P₁V₁=P₂V₂ where: P₁ is the initial pressure; V₁ is the initial volume, P₂ is the final pressure and V₂ is the final volume. Therefore for a gas at a constant temperature, pressure×volume is a constant. So increasing pressure from pressure 1 to pressure 2 means that a volume incurs a consequent reduction, providing the temperature remains constant.

Whilst the arrangements illustrated in FIGS. 1 to 3 include three media chambers, it will be appreciated that additional media chambers could be integrated, based upon correct valve balance and pressure release points. Inclusion of further media chambers may facilitate cell culture devices and systems which support multi-organ/tissue/cells integration.

Components of arrangements may be configured such that the pressure valves for fluid servicing operate at a higher pressure compared to the valves provided to support fluid circulation. Such a configuration may prevent a return flow of fluid into the service tank when the fluid circulation is in action, and may help to create a very secure directional flow in the system.

Breather/pressure equalising vent valves may be provided in support of both fluid servicing and fluid circulation. Vent valves help to regulate the pressure within the system and prevent choked flow.

Arrangements may provide for integration of one or more sensors and/or probes. According to some arrangements a PCB may be positioned within the device between the liquid and environmental air phase. The PCB may be configured to provide direct connection to electronic circuitry and support operation of, for example, impedance probes and/or liquid temperature sensors provided to monitor operation of components forming the culture environment. Appropriate location of the PCB and selection of appropriate probes and sensors may prevent a need to provide multiple connectors and cables within the operational part of any device. The use of arrangements in which direct, for example, surface to surface, connection between a PCB electrode and a sensor or probe may facilitates integration of sensors within a device and may enable efficient cleaning and assembly/dismantling of a system when a tissue/biological/cell/liquid sample within a device requires access.

A PCB included within arrangements may be coated with conformal coating such that it is protected from the environment and consequent possible corrosion as a result of exposure to the culture environment during experiments. Provision of a PCB coating may help to ensure that the PCB is compatible with humidity and sterilising processes which may include autoclaving, gamma and x-ray irradiation.

Sensors may be integrated into devices according to some arrangements. Sensors and/or probes may be configured to assess one or more environmental condition within liquid and gas phases of a culture environment. Sensors and probes may be configured to monitor and/or assess one or more property indicative of a characteristic of a biological sample under study. For example, impedance probes may be provided to assess tissue (e.g. skin barrier) function and behaviour.

Sensors and/or probes located in the liquid phase of some arrangements may be couplable to the sensor PCB using surface to surface electrical contacts and/or spring contacts. Such configurations may support ease of device assembly and disassembly without the need for cable/wire connections.

Sensors and/or probes may be environmentally sealed within or to a component of a device to ensure the cell culture environmental gas phase remains separate from the liquid phase.

Sample holding:

Arrangements may provide for a suitable biological sample holder within the first chamber. The sample holder may be configured to contain and support the biological sample under study appropriately within a culture environment created by the device in use. In devices which support a biphasic culture environment, the sample holder may be designed to ensure that one surface of the biological culture may be exposed to a gaseous environment while another surface of the biological culture is submerged in the fluid phase. The base of the sample holder may be constructed from a mesh in order to facilitate fluid immersion of one side of the biological culture. A sample plug may be provided to seal the biological culture within a sample holder. The sample plug may be constructed from an appropriate material and located around the periphery of a biological sample to be studied such that fluid gas phases of a biphasic culture environment supported by devices do not mix. In biphasic culture arrangements, a sample holder is positionable between the gas phase and an uppermost fluid phase chamber.

Devices according to various arrangements support transport and portability of biological samples locatable within the device for study. Arrangements may be provided in which all chambers and/or layers are environmentally sealed such that there is no liquid or gas exchange between layers. Arrangements may be such that pressure valves which control media exchange between chambers in operation remain closed during transport since the valves are configured to remain closed under ambient pressure conditions.

In arrangements in which biphasic culture is supported, use of a sample plug seals the biological culture in a sample holder between fluid chambers and environmental gas chambers so that biological samples can be transported in the chamber of the device, with media, without the media leaking. The biological culture may, as described in relation to the sample holder, be positioned on a mesh inside a well. In some arrangements a sealing plug can be used, for example, by pressing the seal against the top of the tissue, thereby creating a seal with the tissue itself. A sealing flange around the outside diameter of a sealing plug can be used to create a seal between the fluid and gas phase of the system. A simple frictional push fit of a sealing plug can maintain an appropriate seal and allows for ease of insertion and removal.

As described generally in relation to apparatus in FIG. 4, arrangements may provide for systems in which gas phase control is enabled. The device may include an air phase port, couplable to a source of environmental gas. In some arrangements, the environmental gas may comprise filtered air. Some apparatus arrangements may be such that a control unit is configured to provide sterile filtered air to the air phase of a culture environment. Sterile filtered air may enter apparatus from a central port in an environmental chamber. A lid, or cover, provided according to some device arrangements may be configured to channel filtered air into separate streams, such that the air provided flows in a known flow pattern selected to prevent atmospheric mixing between units. In some arrangements, a gas permeable filter or barrier such as a filter material which is gas permeable but prevents the passing of liquid and particulate matter can be included in each unit gas inlet. The filter or barrier may be configured to filter incoming gas. If filtration of provided by a gas permeable barrier is not adequate for a particular application then additional air filter pads can be added.

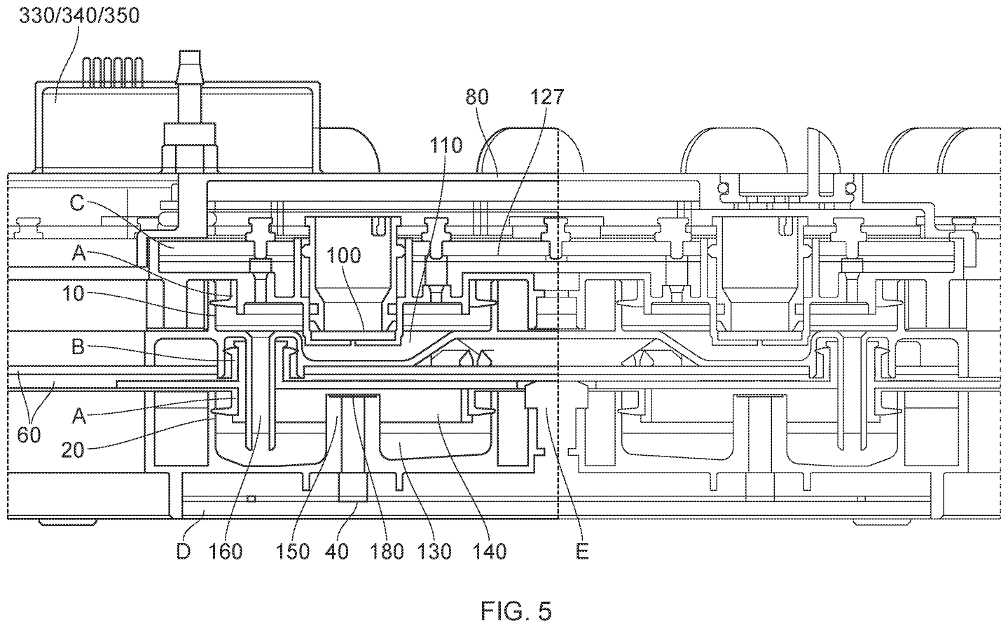

FIG. 5 illustrates schematically in cross section an alternative embodiment of some main components of one possible arrangement relating to circulation of fluid within a multi-unit module. In relation to the embodiment shown in FIG. 5, like reference numerals have been used for like parts as referenced in relation to FIGS. 1 to 4.

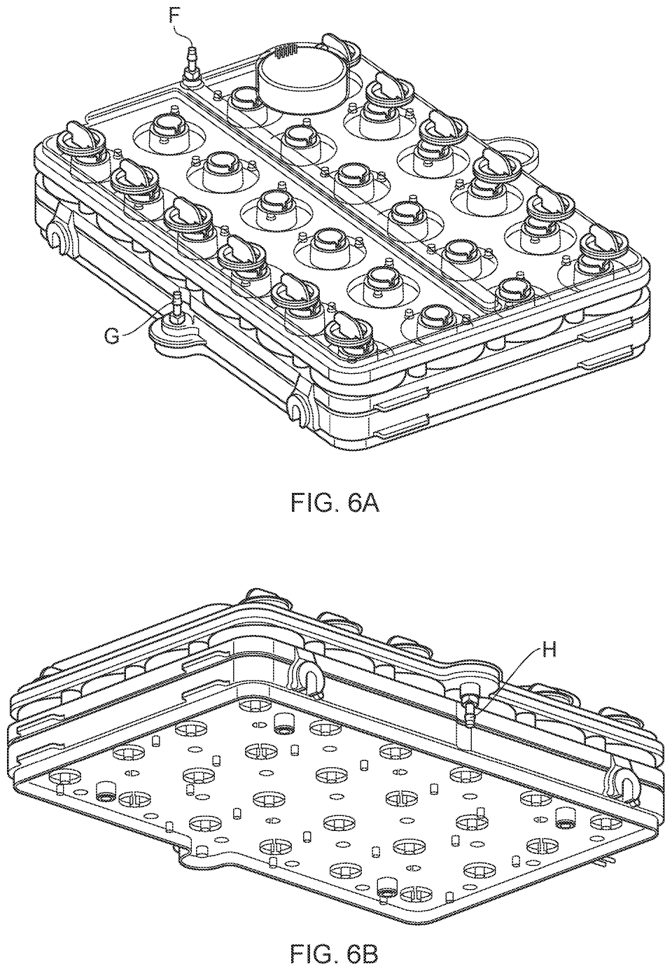

The arrangement shown in FIGS. 5 and 6 comprises: a large wiper seal A arranged to help form chambers 10 and 20 between the body and lid parts. Wiper seal comprises an interface seal to help the manufacture of chambers 10 and 20. Wiper seal A may comprise a wiper seal (as shown) or, in some arrangements, a face (flat) gasket.

The arrangement shown in FIGS. 5 and 6 comprises: a smaller wiper seal B which is configured to form a seal for inlet conduit 160 to pass through the heating plate 60.

The arrangement shown in FIGS. 5 and 6 comprises: a seal C in the form of Polydimethylsiloxane (PDMS) encapsulation arranged to form a seal in relation to exhaust path 127.

The arrangement shown in FIGS. 5 and 6 comprises: a seal D in the form of Polydimethylsiloxane (PDMS) encapsulation which replaces items 90 from FIG. 1 to create gas channel 40.

The arrangement shown in FIGS. 5 and 6 comprises: a silicone bung E configured to to provide an upward force for heating plate 60 against the bottom of first chamber 10.

FIGS. 6A and 6B are isometric representations of some of the main components of a multi-unit module according to an alternative arrangement such as the one shown in

FIG. 5. Gas entry F for the air phase into the module is shown, together with gas entry G for gas channel 40. Similarly, Gas exit H for exhaust path 127 is shown. It will be appreciated that gas pressure regulation chamber 120 and sealing plug 125 may be located outside the unit, as shown in relation to FIG. 1.

Aspects and embodiments of devices and methods described herein may have particular application in relation to cell culture arrangements which are capable of maintaining viability, in an ex vivo environment, of various biological samples. Particular application may be found in relation to biphasic cell culture arrangements in which it is desired to culture cells, cell populations and/or tissue which is typically exposed to a liquid/gas environment. For example, tissue which, in a body, would be exposed to environmental atmosphere (skin, eye, oral epithelia).

Systems using devices and methods described herein may be suited to cell culture systems which support one or more of: scaling; automation of media flow and/or harvesting; long term maintenance of a biological sample under study, for example, tissue such as skin; integration of testing analytics; applications for combining various biological sample assays, for example, skin absorption, corrosion, irritation, sensitisation within one system; repeated dose toxicity; pharmacokinetics; and/or prediction of sample response to topically applied products.

Although described in relation to a biological sample culture unit, it will be appreciated that the principles and features described in relation to the biological sample culture unit may be applicable to a more general fluidic device. Accordingly some arrangements provide a fluidic device comprising a first chamber configured to accommodate a fluid, the first chamber including a pressure equalising valve which vents to a surrounding environment; a second chamber configured to accommodate a reservoir of fluid and a gas, the second chamber comprising a gas port couplable to a gas pump; an inlet conduit linking the second chamber with the first chamber comprising a one way valve configured to allow flow of fluid from the second chamber to the first chamber when pressure difference between the second chamber and the first chamber exceeds a preselected inlet threshold; and an outlet conduit linking the first chamber with the second chamber comprising a one way valve configured to allow flow of fluid from the first chamber to the second chamber when the pressure difference between the first chamber and the second chamber exceeds a preselected outlet threshold.

Some arrangements provide a method of arranging and forming components to provide such a fluidic device.

It will be appreciated by those skilled in the art that the various devices described in accordance with arrangements support various novel biological test modalities and regimes. Features of many of those modalities and regimes are referenced throughout the above description. Accordingly, some aspects of the invention may provide a method of culturing a biological sample comprising: locating the biological sample and a culture medium within a first chamber of a biological culture unit, the first chamber including a breather valve; providing a reservoir of culture medium and a gas within a second chamber, the second chamber comprising a gas port couplable to a gas pump; providing an inlet conduit linking the second chamber with the first chamber, the inlet conduit comprising a one way valve configured to allow flow of fluid from the second chamber to the first chamber when pressure difference between the second chamber and the first chamber exceeds a preselected inlet threshold; and providing an outlet conduit linking the first chamber with the second chamber, the outlet conduit comprising a one way valve configured to allow flow of fluid from the first chamber to the second chamber when the pressure difference between the first chamber and the second chamber exceeds a preselected outlet threshold.

The devices described may have particular utility in relation to the study of biphasic biological samples, for example skin cells, skin tissue and similar, as described above.

Methods using the device support test regimes which allow full automation and/or continuous monitoring. Test methods implemented may be such that there are no fixed endpoints increasing biological testing rates.

The device provides a mechanism to support provision of alternative and differing liquid-phase media chemistry, for example, to mimic circadian rhythms, and/or provide timed drug/compound delivery and monitor for responses; and or to interrogate hormonal responses, and/or to provide timed immune cell delivery.

The device provides a mechanism to support provision of accurate oxidative stress-analysis (in a biphasic system) for example, as a result of a method which implements media changes without disturbing internal oxygen levels, whilst also maintaining apical/environmental oxygen/CO₂ levels.

The device provides a mechanism to support methods which mimic and/or seek to improve human skin sun protection factor testing. The device enables a methodology in which sunscreen can be placed on a skin sample, the sample can be exposed to appropriate electromagnetic radiation, and testing for skin and DNA damage may be achieved over a period of, for example, days. Undisturbed environmental control facilitated by device arrangements means that oxidative stress from UV irradiation can be accurately measured.

The device provides a mechanism to support toxicology testing methods. For example, the device allows for measurement of, for example, cell death and/or changes in metabolism under a controlled environment over an extended period. Arrangements allow maintenance of a biological sample culture environment in which media changes are unlikely to interfere with cell behaviour.

The device provides a mechanism to support improved maintenance of cell types, for example, stem cells, that may be more prone to environmental fluxes, by facilitating control of a biological sample culture environment.

The device provides a mechanism to support simultaneous testing, for example, corrosion, irritation, of a biological sample in formulation by providing for in vitro repeat-dose testing. The device provides a mechanism to support in vitro methods which mimic in vivo human patch testing.

The device provides a mechanism to support Raman imaging of biological samples to support multi-parameter, real-time (skin) testing.

The device provides a mechanism to support test methods which can account for interperson-variation. The mass testing facilitated by devices allows for use of sensors to determine differences in human populations, for example, in relation to biological samples comprising skin samples. Data captured in a mass-sample environment can generate data which can be coupled with machine learning/AI to recognise previously undetected patterns or features associated with a type of biological sample.

According to a further aspect, there is provided a test plate comprising: at least one chamber configured to receive a sample, and at least one probe integrated into the test plate and configured to measure a parameter associated with the sample locatable within the chamber, wherein the integrated probe is directly couplable to a contact provided on a printed circuit board placeable adjacent to a surface of the test plate.

According to further aspects, there is provided methods of providing and operating such a test plate.

It will be appreciated that the integration of one or more probes or sensors within a test plate to allow for monitoring, measurement or stimulus of a sample locatable within a chamber of the test plate may be performed in a manner that those sensors or probes can be directly connected to appropriately located electrodes or connectors provided on a PCB. A PCB may then be abutted against the test plate so that a portion or surface of a probe or sensor contacts the appropriately located electrodes or connectors provided on the PCB. Such a simple connection mechanism may minimise complex connections or wires within a device. Connection of a monitoring or control unit to one or more sensors or probes within the test plate may then be achieved by means of a further connector or electrode provided at an accessible portion of the PCB.

The PCB arrangement described and illustrated in relation to the fluidic device of some aspects may be provided without the fluidic aspects to arrive at a test plate according to the further aspect.

A person of skill in the art would readily recognize that some steps of various above-described methods can be performed by programmed computers or microcontrollers. Herein, some embodiments are also intended to cover program storage devices, e.g., digital data storage media, which are machine or computer readable and encode machine-executable or computer-executable programs of instructions, wherein said instructions perform some or all of the steps of said above-described methods. The program storage devices may be, e.g., digital memories, magnetic storage media such as a magnetic disks and magnetic tapes, hard drives, or optically readable digital data storage media. The embodiments are also intended to cover computers programmed to perform said steps of the above-described methods.

Although embodiments of the present invention have been described in the preceding paragraphs with reference to various exemplary arrangements, it should be appreciated that modifications to the examples given can be made without departing from the scope of the invention as claimed.

Features described in the preceding description may be used in combinations other than the combinations explicitly described.

Although functions have been described with reference to certain features, those functions may be performable by other features whether described or not.

Although features have been described with reference to certain embodiments, those features may also be present in other embodiments whether described or not.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. A biological sample culture unit comprising:
a first chamber comprising a first chamber volume configured to accommodate a biological sample, a culture medium, and a first gas reservoir;
a second chamber comprising a second chamber volume configured to accommodate a reservoir of culture medium and a second gas reservoir, the second chamber comprising a gas port configured to couple the second gas reservoir to a gas source; and
an inlet conduit extending between the second chamber and the first chamber configured to allow flow of fluid between the second chamber and first chamber in dependence upon a pressure difference inducible between the second chamber and the first chamber.

2. The biological sample culture unit according to claim 1, wherein the inlet conduit is housed entirely within the first chamber and the second chamber.

3. The biological sample culture unit according to claim 1, comprising a gas pressure regulation chamber coupled to the first chamber volume via a first chamber exhaust configured to allow restricted flow of gas between the first chamber volume and the gas pressure regulation chamber.

4. The biological sample culture unit according to claim 3, wherein:
the first chamber exhaust comprises a dog leg conduit; and
the first chamber exhaust has a narrow diameter compared to a diameter of the inlet conduit extending between the first chamber and the second chamber.

5. The biological sample culture unit according claim 1, further comprising a biphasic biological culture unit comprising a biological sample holder located within the first chamber, the biological sample holder being configured to accommodate the biological sample such that a first surface of the biological sample is exposed to the culture medium and a second surface of the biological sample is exposed to an environment outside the first chamber.

6. The biological sample culture unit according to claim 5, further comprising:
a lid, the lid being configured to enclose a portion of the first chamber and to provide a closed environment outside the first chamber to which the second surface of the biological sample locatable within the biological sample holder may be exposed, and wherein:
the closed environment comprises a gaseous environment; and
the lid comprises an inlet configured to receive compressed air into the closed environment and an outlet configured to allow escape of gas from the closed environment.

7. The biological sample culture unit according to claim 1, comprising a sensor configured to monitor a parameter indicative of a characteristic of the biological sample within the culture medium, and wherein:
the first chamber comprises an opening configured to receive at least a portion of the sensor; and
the sensor is attached with a direct coupling to a PCB locatable immediately adjacent to a wall of the first chamber, wherein the direct coupling comprises a surface to surface conductive connection.

8. The biological sample culture unit according to claim 1, wherein the gas port comprises a gas permeable, fluid impermeable valve.

9. The biological sample culture unit according to claim 1, further comprising:
a third chamber configured to accommodate a source of culture medium and a service gas reservoir, the third chamber comprising a further gas port couplable to a gas pump;
a service inlet conduit linking the third chamber with the second chamber comprising a one way valve configured to allow flow of fluid from the third chamber to the second chamber when a gas pressure difference between the third chamber and the second chamber exceeds a preselected inlet threshold; and
an outlet conduit linking the second chamber with the third chamber comprising a one way valve configured to allow flow of fluid from the second chamber to the third chamber when a pressure difference between the second chamber and the third chamber exceeds a preselected outlet threshold.

10. The biological sample culture unit according to claim 9, wherein the third chamber comprises at least two well portions a first well portion configured to receive the source of culture medium, and from which the service inlet conduit extends into the second chamber, and a second well portion configured to receive a flow of fluid from the second chamber through the outlet conduit; and
wherein the at least two well portions are at least partially separated by a barrier permeable to gas, operable to prevent liquid flow between the at least two well portions.

11. The biological sample culture unit according to claim 1, wherein:
the biological sample culture unit is configured such that the biological sample culture unit and an additional biological sample culture unit can be placed into a biological culture module; and
the biological culture module comprises a gas distribution member configured to couple to the gas port of each biological culture unit and a source of gas.

12. The biological sample culture unit according to claim 11, wherein:
the gas port of each biological culture unit comprises a port valve configured to allow flow of gas to or from the gas source into the second chamber of each biological culture unit;
the port valve of each biological culture unit operates simultaneously upon exposure to a flow of gas to or from the gas source; and
the port valve is configured to allow flow of gas out of the second chamber.

13. The biological sample culture unit according to claim 11, wherein the biological sample culture unit comprises a third chamber, the biological culture module comprises a further gas distribution member configured to couple to a further gas port of each biological sample culture unit and a gas pump.

14. The biological sample culture unit according to claim 13, wherein the further gas port of each biological sample culture unit comprises a port valve configured to:
allow flow of gas from the gas pump into the third chamber of each biological sample culture unit when the pressure difference exceeds a first threshold; and
allow flow of gas from the third chamber of each biological sample culture unit towards the gas pump when the pressure difference exceeds a second threshold.

15. (canceled)

16. The biological sample culture unit according to claim 1, further comprising a control unit, configured to control flow of gas into the second chamber to circulate the culture medium locatable within the first chamber and the second chamber.

17. The biological sample culture unit according to claim 16, wherein the control unit is configured to implement a regular periodic flow of gas into the second chamber to move culture medium between the first chamber and the second chamber.

18. The biological sample culture unit according to claim 16, wherein:
the biological sample culture unit comprises a third chamber, and
the control unit is configured to control flow of gas into and out of the third chamber to exchange culture medium locatable within the second chamber and the third chamber.

19-22. (canceled)

23. A method of culturing a biological sample comprising:
locating the biological sample, a culture medium and a first gas reservoir within a first chamber of a biological culture unit, the first chamber having a first chamber volume;
providing a reservoir of culture medium and a second gas reservoir within a second chamber having a second chamber volume, the second chamber comprising a gas port configured to couple the second gas reservoir to a gas source;
providing an inlet conduit extending between the second chamber and the first chamber, the inlet conduit configured to allow flow of fluid between the second chamber and the first chamber in dependence upon a pressure difference inducible between the second chamber and the first chamber.

24. The method of culturing the biological sample according to claim 23, further comprising applying a flow of gas to the gas port of the second chamber to increase gas pressure in the second chamber and circulate culture medium from the reservoir of culture medium in the second chamber to the first chamber.

25. The method of culturing a biological sample according to claim 23, further comprising preventing a flow of gas to and from the gas port of the second chamber to allow a gas pressure differential induced between the second chamber and first chamber to equalise, resulting in a transfer of culture medium from the first chamber to the second chamber through the inlet conduit.
